SEQUENCE LISTING (56 sequences)

SEQ ID NO: 1 - DNA, 9890 bp, Homo sapiens
acaagctctccatgtgagctgacaggcgagtggaaacccctcgagtcacgctgcccggcggcggagggagcgctcgcccgcagtggcaacagctgcaccaccgtccccgtcgctctgccttcctcttctgcagcctctgctcttctgattacctccctcccccgtcctttggtgatttttttttttcaagaaggagagggcggggtaggtgtccgttccctcccctcttccccctcctttgccttcttggtttgaatttcctcccccggcgttgcactggcacacagtgcaagaggcaatacccgcacggagggagaacgaaggctgagactcccctgccgctccaagcccggaagaactggagcctggaggggggtgaggggagaagaggaagcgggaggggcttggcttcctcgcgtatttgaggacagcccatctcccttcaagaaccctacggagagtcggactgcatctccgcagcgagctcttggagcgccgccggccgggaggcgaaggatgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaactttaagacgtctgaaagtgaaggagtaatcctgcacggagaaggacagcaaggagattacattaccttggaactgaaaaaagccaagctggtcctcagtttaaacttaggaagcaaccagcttggccccatatatggccacacatcagtgatgacaggaagtttgctggatgaccaccactggcactctgtggtcattgagcgccaggggcggagcattaacctcactctggacaggagcatgcagcacttccgtaccaatggagagtttgactacctggacttggactatgagataacctttggaggcatccctttctctggcaagcccagctccagcagtagaaagaatttcaaaggctgcatggaaagcatcaactacaatggcgtcaacattactgatcttgccagaaggaagaaattagagccctcaaatgtgggaaatttgagcttttcttgtgtggaaccctatacggtgcctgtctttttcaacgctacaagttacctggaggtgcccggacggcttaaccaggacctgttctcagtcagtttccagtttaggacatggaaccccaatggtctcctggtcttcagtcactttgcggataatttgggcaatgtggagattgacctcactgaaagcaaagtgggtgttcacatcaacatcacacagaccaagatgagccaaatcgatatttcctcaggttctgggttgaatgatggacagtggcacgaggttcgcttcctagccaaggaaaattttgctattctcaccatcgatggagatgaagcatcagcagttcgaactaatagtccccttcaagttaaaactggcgagaagtacttttttggaggttttctgaaccagatgaataactcaagtcactctgtccttcagccttcattccaaggatgcatgcagctcattcaagtggacgatcaacttgtaaatttatacgaagtggcacaaaggaagccgggaagtttcgcgaatgtcagcattgacatgtgtgcgatcatagacagatgtgtgcccaatcactgtgagcatggtggaaagtgctcgcaaacatgggacagcttcaaatgcacttgtgatgagacaggatacagtggggccacctgccacaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttgaggggtggctacttggctatgggatagggaggagggaattactagggaggagagaaagggacaaaagcaccctgcttcatactcttgagcacatccttaaaatatcagcacaagttgggggaggcaggcaatggaatataatggaatattcttgagactgatcacaaaaaaaaaaacctttttaatatttctttatagctgagttttcccttctgtatcaaaacaaaataatacaaaaaatgcttttagagtttaagcaatggttgaaatttgtaggtactatctgtcttattttgtgtgtgtttagaggtgttctaaagacccgtggtaacagggcaagttttctacgtttttaagagcccttagaacgtgggtattttttttcttgagaaaagctaatgcacctacagatggcccccaacattctcttccttttgcttctagtcaaccttaatgggctgttacagaaactagttcgtgtttatatactatttcctttgatgtcctataagtcggaaaagaaaggggcaaagagaacctattatttgccagtttttaagcagagctcaatctatgccagctctctggcatctggggttcctgactgataccagcagttgaaggaagagagtgcatggcacctggtgtgtaacgacacaatcagcacaactggagagaggcattaaagaaccagggaaggtagtttgatttttcattgaattctacaagctaatattgttccacgtatgtagtcttagaccaatagctgtaactatcagctgcaataccatggtgaccagctgttacaaaagattttttcctgttttatctgaaacatactggatttatatatgtataagcgcctcaatggggaattagagccagatgttatgatttgtttgctctttttcttttatagtttagttatagcaaaaatatggataatttctagtgaatgcataaattaggttgcgtttcttattttgctttaaatctctggtagtttttccacccctgtgacacaatcctaatagacagtgtcctgtaaatggacacaacacaataaagtcaagttattattgctgttactctggatgatatggaaaacactgccatattttaaatcaactactccacgtgtttttccatccaatcacactgctgtgattcagggatctttcttctaagacggacacatttgaacctcaggttcatcacaaacctggtacctgttgcttcccagaggatggagaagtgtagttaatcacacctcttagtttaatctgaaatcttgacccagttatttaacaaataaatacctcattgattatatttaaaagtaatacacttcctgtaaacaaatggggacaatgcatccaaaaaatctttttaaacagattacacaaaaattatttccagaaaggctaccatttatcatcattatatttcaagcctcttatacttaataagcactttctaaaaagtcttgagatcccaccattctgaggaattcaatatgatcactttttccttctttgcctgggagaggttaagaggaggtttcgaaggtatagatgctattgttctgatggcccggctgaataaaatggaaattctagtttgttagaattatgcattctttttcaagattctcagtgtgcctaacttattggagcacatcagtttcttgggtaatggaaaacattacctagagttgccagtggcacattacaccagtacagagcacattccaaaggagacattggaccagttaattcccatacaagtcaaggtaacagaacaaaagggaatcctgatgcccttttaccattgctggttgagctcaggcactgtcatggacacccttaattttaaaaggttttaatcattcttctataaaatacatttaaaatggaaaaatacttaatatcactaaatatcagaacaatgtaacatttacaaatgacatattgaaagcaaaggctgttttatttagccaagatgattaccattaggagttactttatgtattgttgaaagcaaattttaaacatgatgttttagaagtgtttctgatttttaaacctggtttacaggtattacttctgcacttaccaaataatgccagatggaaatttattatttcttgcaattcccatgatagctctgttctttatgcattgtctcaacactttcccttttttcccaaaatgagtagagaattaaagccacccaaaacagcttctgctactaaaatgttctcatcctttctcctccctctccttttcctgccacaaaaggtgaaaaatgagatccaatcctctcaccaaaatttcaaacctaggacactggaatgactgcagggatcagtggttctcccatatcaccatcaattaagacatataggacactgtcttccttcaagagggttacaatgtggccatcagacaggaaaccaaacggtggataaagtattaagtaactaagtgccaaataaatgctggaaatcttgacctctccttgggattatgggtgtaacaaaaatccctacatctgtttatgaaggccatattcagtacattttaaatggtaaataatctgtttatgtgaagaaaaagaattaagtctttcttccaactctctccttggatagcctagcacagtgcagcctccataaccatgacattcccgcccaagctctcagtgcctaatcctgctttgtcattcacatctcacaaaatcttgacatcttacattccaatacattatcaagcaagcacaagtatgctggtagtagcctctttaaataatatgtatagacaacaacaacgacaaaaaatagactgttttaaagtttcagggaaagttggtggctgatttaaagttgtgcaggaaacatcttctgtgtatgaagcaaatgtcgatgttttgaaaaaagctaggagatgactttgaatgaatgcaaggttagtgagatcctaagctctcaaaatagcatattccctagagctcaagaaagctggtccaggaggttgaaaaagctattttgttgttaaattattttctggcccttcttaatatttaaaaatgtatttccccttgtggctttcaaccacctgctcaaaaaaagagacttgttacatgaaagttttcattaaagagctgaaaacaagaatttagagagccattcctagaaaatgtcctactgccctgcatttgacaaacaagcatcctttactaacaagagcaggaattcagaggcacaagaaaaagcattggcatgagccaaagagtctgtcttaatgttacttttgaaaatctgctgagcggccaccatatgcaggctgagagctgggcacaggcgaagccattggaagcacttcaggaacaagcacacagctgtgggacttgaacatgcaagtgttcaggttgtgtcaagaagcttttctttccttctatgatggaatctgttcttttctatcctacttttttctctcttcctctcctcaccacattataccctgctcttacgcagtaaacgttttaatggcccgtttatgtctcatgcctccaaacaacactgaatttgaaaccccccattttttcttttcaccaccctgttgagcaattttcccaaaaaaagggcagcaattattaaattgaattcaagtaagccagccaaagataggtcctaaattgctagtcccagtagaaccacctgatcctaaaccagtgcgaaacaaacagtaacaatgtccccagctgacttcagctaagaaccaatggctcctacccccgccccgcttttttttgttgttttttgttttgttttgagacggagtcttgctctgtcccccaggctggagtgcactggcgcaatctcgggctcactgcaacctcctcctcctcccacattgaggcgattctcctgcctcagcctcccaagtagctgggattacaggcacccgccatcacacccagctaatttttttttttttttgtattattagtagaagccaggtttcaccatgttggccagggtggtctcgaactcctgacctcaagtgatccgtccacctcggccttgcaaattgctgggattacaggtgtgagccaccgtgccgagccagccccattttttaaatgatgttttggttaagagtggaccatgagaattagctgacagcatcccctttctctctccctgccttggtgggaccctccctgtgtgaccttggtcaagtcctcgaacttttgtcccgtatttaagatggagctgttttacctacttcataagacagttgcgaggtgccattgattcttgactgcaaaataccttgaaacccttatataaagactgaagtcaacggagcctagtgaaagacttactttgtggcttgtggttgaaagtcacatcaaaagacaaatgtggccacgttcaggaattggagacttactggcatggctctacagctgctcagttattaatcatgcagactaacctgtcaacactgggagatgcaacatagcaaaaggacagagaaattagaattttttgtgcagaaagccctaaattcccacctgaatgtaacttacagctcccttacctactctcacacatgccctcaaacatgctagattggcttatacataggccaacacaaaatacaaacgtgacgtgttcatgtagcctagtggctatatgcctattctccatgtaccctgcatggtagtgctgcaaactttaaagtacatttctttcacagcagtattttttttcataagtggcatataaatgtcattcaatgaaatggggaaatcacgttgagaagttggtctgtcatctcccattgagcaaagactggcaggagataataaaaataaatatgggcacacatgtattaatatacagcacgcatttacaagtttattttccagataaaattgtgctataagaacagctctaccaagacagtctgcaccatttccaagtctcagttaatttacagcaactgctgctttcggagatggctgtgaaaatatggaagttcctctcaagtaggccaagaaacagttctagattttactaagttttattttgtcaggttttttaaattttttcagtgagcgtggtgactgcagaggttagtgctgtgaaaagctgggctaaatattctttctgtaaagtcaaacaggattccatcccctgtgaaataacacaaaatttcactctctaaaagcaacagcatgtaaactagaatgaaagaaggaaattatgtacgtatgcctaatattctttgtgaatgtctttcatttaactaaaattatattagaaaccagattgataaataaaaaattcaaagtagttttaattatcct

SEQ ID NO: 2 - protein, 1331 aa, Homo sapiens
MQAAPRAGCGAALLLWIVSSCLCRAWTAPSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNFKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSCVEPYTVPVFFNATSYLEVPGRLNQDLFSVSFQFRTWNPNGLLVFSHFADNLGNVEIDLTESKVGVHINITQTKMSQIDISSGSGLNDGQWHEVRFLAKENFAILTIDGDEASAVRTNSPLQVKTGEKYFFGGFLNQMNNSSHSVLQPSFQGCMQLIQVDDQLVNLYEVAQRKPGSFANVSIDMCAIIDRCVPNHCEHGGKCSQTWDSFKCTCDETGYSGATCHNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 3 - DNA, 6426 bp, Homo sapiens
gggagctgcgctcgcagtttcgccctctcttccgctaatgattgcattattatgctcccctctctggggggtctcgcccctcttgggtcgctccggagccccggcctcccctggctgcatttcttaaaaatttgggagcctgggagtgagttttctccgaggcgtgtgtgagaggcggcgggggtgttttcctgcgcgaggggcgggtgaagttcattgcccccacttttcccgcgacctttttcggacccgattttggatcgagttgaggggggcgcgggcgttttcggggggcggggggcgcggcggagaatggccgcggggagggctccccggagcctcccagtctcttgatcaaagcattccgctattctgatttattgcttgcttggtgagttatttttttttcctctaaaggagacctgtgtgttcagccattactttgctcggcgctgctcccaggcatctccgaccctcggtgctgtggggagccccacacttgggctcctcgcctctcgccctcgctccccgtccctcctcccctctctccgccccttcccccttttctttctcctctctttcttcccctctctcccttctttcggccgccgtctcccccgcgccctcctcggggcggagggaagccgtgaagggggagggagggctcggtgtcaatttttttttgtgtggctgcggccgtagcctgtggcgggcaagcggggagaccccggcgcagcagaaccatggatggcccgacgcggggccatggactccgcaaaaagcggcggtcgcggtcgcagcgagaccgggagaggcgctcccggggcgggctgggggccggcgcggccggcggcggcggggctggccggacccgggcgctctcactcgcctcgtcgtcgggctccgacaaggaagacaatgggaagcccccgtcctccgccccgtcccggcccagacccccgcggaggaagcggagagagtccacctcggcagaagaggacatcattgatggatttgccatgaccagctttgtcacttttgaagcgctggagaaagatgtagcacttaagcctcaggaacgtgtggagaaacgccagacgcccctgaccaagaagaaacgagaagcacttaccaatggcttgtcctttcattcaaagaagagcagactcagccacccacaccactacagctcagatcgagaaaatgaccgcaatctctgccagcaccttgggaagagaaagaaaatgccgaaggcactcagacagctcaagccaggacagaacagctgcagggacagtgacagtgaaagtgccagtggagaatccaagggcttccaccggagcagctctcgggaaaggctcagtgatagttcagctccttccagcttgggaacaggctacttctgtgacagtgacagtgaccaggaagagaaggcatcagatgccagctctgaaaaactcttcaacactgttattgtaaacaaagatccggagttaggtgttggcacgctaccagaacatgacagccaggatgcagggccgattgtccccaagatatcgggtctagagagaagccaggagaagagccaggactgttgcaaagagccaatctttgagcctgtggtgcttaaagacccctgccctcaggtcgcacagccaataccccagccgcagacggagccccaactccgagctccttctccggaccctgacttggtgcagcgcacagaggccccacctcaacccccacctctgagtacacagccaccacagggccctcctgaggcccagctccagcctgccccgcagcctcaggtgcagaggccacccaggccacagtcccccacccagctgctccatcagaacctcccacctgtgcaggcccacccctctgctcagagcctctcccagccattgtcagcctacaacagcagtagcttaagcctcaacagtttaagcagcagcagaagcagcactccagcgaagactcagcccgccccacctcacatctcccaccacccctctgcctccccgttccccctctccctgcccaaccacagccccctgcacagcttcacacccaccctccagccccccgcacactcacatcaccccaatatgtttgcccctcccactgctctgcctcctccaccaccactgacatcaggaagtctgcaggtggccggacacccggccgggagcacttactcagagcaagacatcttgcgacaggaactgaacactcgttttttggcctctcagagtgctgaccgcggggcttccctgggccctccgccctacctgcggaccgagttccatcagcaccagcaccagcaccagcacacccaccagcacacgcaccagcacaccttcacgccgttcccccacgccatcccacccaccgccatcatgccgacgccagcacctcccatgtttgacaaataccctacaaaagttgacccattctaccggcacagtctcttccattcctatcctcctgcagtgtcgggcatcccccctatgatcccacccactggcccttttggttcactacaaggagcatttcagccgaagacatccaaccctatcgatgtcgctgctcggcctgggacagtcccacacactttactccaaaaggacccgaggttgacagatcctttcagacctatgttaaggaaaccagggaagtggtgtgctatgcatgttcacatcgcctggcagatttaccaccaccaacagaaagtcaagaaacagatgcagtcagacccacataagctggactttggactgaaacctgagttcctgagccgccctccaggccccagtctttttggagccatccaccacccccatgacctggcacggccttcaactttgttctctgccgctggtgctgcacacccaactgggaccccttttgggccacctcctcatcacagcaacttcctcaaccctgctgcccacctagagccttttaatcggccgtctacattcacaggcctagcagcagttggtggcaatgccttcgggggacttggaaatccttccgttacacccaactcaatgttcggccacaaggatggccccagtgtgcagaactttagcaaccctcacgaaccctggaaccggctgcaccgaacgcctccgtcgttcccgacccctccgccctggctgaagccaggggagctggagcgcagcgcgtccgctgcagctcatgacagagatagagatgtagataaacgagactcatctgttagtaaagatgacaaagaaagggaaagcgtcgagaagagacactccagccacccttcaccagcacctgtcctcccggtgaatgccctgggacatacccgcagctccactgaacagatccgggctcatctgaacactgaggctcgggagaaggacaaacccaaagagagggagagagaccactcggaatcccgcaaggacctggccgccgacgagcacaaggcgaaagagggccacctgcccgagaaggacgggcacggccacgaggggcgcgccgcgggcgaagaggccaagcagctggcccgggtgccgtctccctacgtgcggaccccggtggtggagagtgccaggcccaacagcacctcgagccgggaggccgagccgcgcaagggtgagccggcctacgagaaccccaagaagagctccgaggtcaaggtgaaggaggagcggaaggaagaccatgacctgcctccagaggccccgcagacccaccgggcctcggagccgccgcctcccaactcctcgtccagcgtgcacccggggcccctggcctcgatgcccatgacggtgggggtgacgggcattcaccccatgaacagcatcagcagcctggacaggactcgcatgatgacccccttcatgggcatcagccccctcccgggcggagagcgcttcccgtacccttctttccactgggaccccatccgggaccccttgagggatccttaccgagaacttgacattcaccggagagacccgctgggcagggacttcctgctaaggaacgacccgctccaccggctctcgactccccggctgtacgaagccgaccgctccttcagggaccgggagcctcacgactacagccaccaccaccaccaccaccaccacccgctgtctgtggaccctcggcgggagcacgagcggggaggccacctggacgagcgggagcgcttgcacatgctcagagaagactacgagcacacgcggctccactccgtgcaccccgcctccctcgacggacacctcccccaccccagcctcatcaccccgggactccccagcatgcactatccccgcatcagccccaccgcgggcaaccagaacggactcctcaacaagacccctccgacagcagcgctgagcgcacctcccccgctcatctccacgctggggggccgcccggtctctcccagaaggacgactcctctgtccgcagagataagggagaggcccccttcccacacgctgaaggatatcgaggcccgataagccgagaacaggagcaagaacgaggaagaagaaaccctaggcagacaccaggccaggcttgagagacagaactcctgcatggctcacacagactgggggggaaagccccaccccttccccttgtaaaaaatgtatagactcagtgcacattttgaaatgttttgtatattatatgttgagatttttcagatcttttagcccagtcatatgttctcacgtctcctactttttgtttctcgtataaaactttttgatttgaaccaaaacagtgaagatgacaacacacaccaattggatgataattgtagcgggggcggtgggggggagaagtccacgccatccatcatgcaaaattctttcagatgaggtgggaaggccgtgtacatagttatgtaaaaagagattgcttcatgagctaatggttcatatatgcaaaagggtaagatgaaagctttactttgtacaaatgtaaatagataaagtaacataatacattaatacttcttaaaatgtgctatttgcaaacttacttaatatcagtgaacacagtcggctaaagctgtgttcccatatattgttatagacagctaaacccttcaactatgcaatgaatgttcgggcttttcacaaaagcccgcctaactcaaaggagccttttcaaatccatttacagcatacttaaggtcatattttccctgaacaagcgcttacgtgatatgactctgttttccttgcttgttttttttcaaacggagaaacatcctgttttgcaaattggaccccaggctggaacttagcatctgaagttgccgcttgtgggctctgggggaaagtgtagccccggagaggtaactgaggacatgagcaaccagtgccagggagggtgggatttgccagatgccaaaatcaggggacgggtggtggtgtctgtcagacacacacaggtcgccagtgacttcacacacacctcatgtgagaaccatgccttttttagtgtgtcctatttcatacctgtacacacttcctcgttttgtaatgagatttacttacacccaaacagatcctgaaagaaagcttcaagttttctcagatgatggatatgttttcactgtattcaataactgacggatgtaaggtgcacgtttcctgatgtgacgcactgtattccagctggtgatcaagtctgggaacagccgtaacaggtcaaccttgtggagccatcgcgagttagagggtgaaagatggcagaaaaaaaagtcttgtgtgtgagtgtgttttttgagtttgcatcaatcttaatgtctcttcataatacttttataatacattaagcctcttgtctacatatttggagagaatatgactttactagcagagaaatacaatatatcttgtctactggactgtaaaatatatgtatgaaataaaattagttccatttggtcttctagtatattaaagtgctatctgacgttgttatcctgtttttgcaaaaaaaaaaaaaaaaaaaagttaactacagaccattgtttctaataagcagagagatctattttagtagtaaactgaaggtttagttgtgagcttcagattttgtgaactccagatgttgtgcggtgtttttttttttttttaagacaacaactaaaaaaaatgcaaggaatatgtacactggaactgtagtggtagctttcagtattgtaaagagattgttctatacggacctttttgctgtttatcctgtatgtaataaagtcctttctagatcctatgtgaaaagaaaagtgaagcaactgaatcttcagcatgttctcatcggcggagccttcttgtgtaatgtaaactgtgccatgttattaaaaaatgtgaactaagcttccagctgcttgtttgtgtgaggtga

SEQ ID NO: 4 - protein, 1259 aa, Homo sapiens
MDGPTRGHGLRKKRRSRSQRDRERRSRGGLGAGAAGGGGAGRTRALSLASSSGSDKEDNGKPPSSAPSRPRPPRRKRRESTSAEEDIIDGFAMTSFVTFEALEKDVALKPQERVEKRQTPLTKKKREALTNGLSFHSKKSRLSHPHHYSSDRENDRNLCQHLGKRKKMPKALRQLKPGQNSCRDSDSESASGESKGFHRSSSRERLSDSSAPSSLGTGYFCDSDSDQEEKASDASSEKLFNTVIVNKDPELGVGTLPEHDSQDAGPIVPKISGLERSQEKSQDCCKEPIFEPVVLKDPCPQVAQPIPQPQTEPQLRAPSPDPDLVQRTEAPPQPPPLSTQPPQGPPEAQLQPAPQPQVQRPPRPQSPTQLLHQNLPPVQAHPSAQSLSQPLSAYNSSSLSLNSLSSSRSSTPAKTQPAPPHISHHPSASPFPLSLPNHSPLHSFTPTLQPPAHSHHPNMFAPPTALPPPPPLTSGSLQVAGHPAGSTYSEQDILRQELNTRFLASQSADRGASLGPPPYLRTEFHQHQHQHQHTHQHTHQHTFTPFPHAIPPTAIMPTPAPPMFDKYPTKVDPFYRHSLFHSYPPAVSGIPPMIPPTGPFGSLQGAFQPKTSNPIDVAARPGTVPHTLLQKDPRLTDPFRPMLRKPGKWCAMHVHIAWQIYHHQQKVKKQMQSDPHKLDFGLKPEFLSRPPGPSLFGAIHHPHDLARPSTLFSAAGAAHPTGTPFGPPPHHSNFLNPAAHLEPFNRPSTFTGLAAVGGNAFGGLGNPSVTPNSMFGHKDGPSVQNFSNPHEPWNRLHRTPPSFPTPPPWLKPGELERSASAAAHDRDRDVDKRDSSVSKDDKERESVEKRHSSHPSPAPVLPVNALGHTRSSTEQIRAHLNTEAREKDKPKERERDHSESRKDLAADEHKAKEGHLPEKDGHGHEGRAAGEEAKQLARVPSPYVRTPVVESARPNSTSSREAEPRKGEPAYENPKKSSEVKVKEERKEDHDLPPEAPQTHRASEPPPPNSSSSVHPGPLASMPMTVGVTGIHPMNSISSLDRTRMMTPFMGISPLPGGERFPYPSFHWDPIRDPLRDPYRELDIHRRDPLGRDFLLRNDPLHRLSTPRLYEADRSFRDREPHDYSHHHHHHHHPLSVDPRREHERGGHLDERERLHMLREDYEHTRLHSVHPASLDGHLPHPSLITPGLPSMHYPRISPTAGNQNGLLNKTPPTAALSAPPPLISTLGGRPVSPRRTTPLSAEIRERPPSHTLKDIEAR

SEQ ID NO: 5 - DNA, 5952 bp, Homo sapiens
cgctcgcagtttcgccctctcttccgctaatgattgcattattatgctcccctctctggggggtctcgcccctcttgggtcgctccggagccccggcctcccctggctgcatttcttaaaaatttgggagcctgggagtgagttttctccgaggcgtgtgtgagaggcggcgggggtgttttcctgcgcgaggggcgggtgaagttcattgcccccacttttcccgcgacctttttcggacccgattttggatcgagttgaggggggcgcgggcgttttcggggggcggggggcgcggcggagaatggccgcggggagggctccccggagcctcccagtctcttgatcaaagcattccgctattctgatttattgcttgcttggtgagttatttttttttcctctaaaggagacctgtgtgttcagccattactttgctcggcgctgctcccaggcatctccgaccctcggtgctgtggggagccccacacttgggctcctcgcctctcgccctcgctccccgtccctcctcccctctctccgccccttcccccttttctttctcctctctttcttcccctctctcccttctttcggccgccgtctcccccgcgccctcctcggggcggagggaagccgtgaagggggagggagggctcggtgtcaatttttttttgtgtggctgcggccgtagcctgtggcgggcaagcggggagaccccggcgcagcagaaccatggatggcccgacgcggggccatggactccgcaaaaagcggcggtcgcggtcgcagcgagaccgggagaggcgctcccggggcgggctgggggccggcgcggccggcggcggcggggctggccggacccgggcgctctcactcgcctcgtcgtcgggctccgacaaggaagacaatgggaagcccccgtcctccgccccgtcccggcccagacccccgcggaggaagcggagagagtccacctcggcagaagaggacatcattgatggatttgccatgaccagctttgtcacttttgaagcgctggagaaagatgtagcacttaagcctcaggaacgtgtggagaaacgccagacgcccctgaccaagaagaaacgagaagcacttaccaatggcttgtcctttcattcaaagaagagcagactcagccacccacaccactacagctcagatcgagaaaatgaccgcaatctctgccagcaccttgggaagagaaagaaaatgccgaaggcactcagacagctcaagccaggacagaacagctgcagggacagtgacagtgaaagtgccagtggagaatccaagggcttccaccggagcagctctcgggaaaggctcagtgatagttcagctccttccagcttgggaacaggctacttctgtgacagtgacagtgaccaggaagagaaggcatcagatgccagctctgaaaaactcttcaacactgttattgtaaacaaagatccggagttaggtgttggcacgctaccagaacatgacagccaggatgcagggccgattgtccccaagatatcgggtctagagagaagccaggagaagagccaggactgttgcaaagagccaatctttgagcctgtggtgcttaaagacccctgccctcaggtcgcacagccaataccccagccgcagacggagccccaactccgagctccttctccggaccctgacttggtgcagcgcacagaggccccacctcaacccccacctctgagtacacagccaccacagggccctcctgaggcccagctccagcctgccccgcagcctcaggtgcagaggccacccaggccacagtcccccacccagctgctccatcagaacctcccacctgtgcaggcccacccctctgctcagagcctctcccagccattgtcagcctacaacagcagtagcttaagcctcaacagtttaagcagcagcagaagcagcactccagcgaagactcagcccgccccacctcacatctcccaccacccctctgcctccccgttccccctctccctgcccaaccacagccccctgcacagcttcacacccaccctccagccccccgcacactcacatcaccccaatatgtttgcccctcccactgctctgcctcctccaccaccactgacatcaggaagtctgcaggtggccggacacccggccgggagcacttactcagagcaagacatcttgcgacaggaactgaacactcgttttttggcctctcagagtgctgaccgcggggcttccctgggccctccgccctacctgcggaccgagttccatcagcaccagcaccagcaccagcacacccaccagcacacgcaccagcacaccttcacgccgttcccccacgccatcccacccaccgccatcatgccgacgccagcacctcccatgtttgacaaataccctacaaaagttgacccattctaccggcacagtctcttccattcctatcctcctgcagtgtcgggcatcccccctatgatcccacccactggcccttttggttcactacaaggagcatttcagccgaagttgacagatcctttcagacctatgttaaggaaaccagggaagtggtgtgctatgcatgttcacatcgcctggcagatttaccaccaccaacagaaagtcaagaaacagatgcagtcagacccacataagctggactttggactgaaacctgagttcctgagccgccctccaggccccagtctttttggagccatccaccacccccatgacctggcacggccttcaactttgttctctgccgctggtgctgcacacccaactgggaccccttttgggccacctcctcatcacagcaacttcctcaaccctgctgcccacctagagccttttaatcggccgtctacattcacaggcctagcagcagttggtggcaatgccttcgggggacttggaaatccttccgttacacccaactcaatgttcggccacaaggatggccccagtgtgcagaactttagcaaccctcacgaaccctggaaccggctgcaccgaacgcctccgtcgttcccgacccctccgccctggctgaagccaggggagctggagcgcagcgcgtccgctgcagctcatgacagagatagagatgtagataaacgagactcatctgttagtaaagatgacaaagaaagggaaagcgtcgagaagagacactccagccacccttcaccagcacctgtcctcccggtgaatgccctgggacatacccgcagctccactgaacagatccgggctcatctgaacactgaggctcgggagaaggacaaacccaaagagagggagagagaccactcggaatcccgcaaggacctggccgccgacgagcacaaggcgaaagagggccacctgcccgagaaggacgggcacggccacgaggggcgcgccgcgggcgaagaggccaagcagctggcccgggtgccgtctccctacgtgcggaccccggtggtggagagtgccaggcccaacagcacctcgagccgggaggccgagccgcgcaagggtgagccggcctacgagaaccccaagaagagctccgaggtcaaggtgaaggaggagcggaaggaagaccatgacctgcctccagaggccccgcagacccaccgggcctcggagccgccgcctcccaactcctcgtccagcgtgcacccggggcccctggcctcgatgcccatgacggtgggggtgacgggcattcaccccatgaacagcatcagcagcctggacaggactcgcatgatgacccccttcatgggcatcagccccctcccgggcggagagcgcttcccgtacccttctttccactgggaccccatccgggaccccttgagggatccttaccgagaacttgacattcaccggagagacccgctgggcagggacttcctgctaaggaacgacccgctccaccggctctcgactccccggctgtacgaagccgaccgctccttcagggaccgggagcctcacgactacagccaccaccaccaccaccaccaccacccgctgtctgtggaccctcggcgggagcacgagcggggaggccacctggacgagcgggagcgcttgcacatgctcagagaagactacgagcacacgcggctccactccgtgcaccccgcctccctcgacggacacctcccccaccccagcctcatcaccccgggactccccagcatgcactatccccgcatcagccccaccgcgggcaaccagaacggactcctcaacaagacccctccgacagcagcgctgagcgcacctcccccgctcatctccacgctggggggccgcccggtctctcccagaaggacgactcctctgtccgcagagataagggagaggcccccttcccacacgctgaaggatatcgaggcccgataagccgagaacaggagcaagaacgaggaagaagaaaccctaggcagacaccaggccaggcttgagagacagaactcctgcatggctcacacagactgggggggaaagccccaccccttccccttgtaaaaaatgtatagactcagtgcacattttgaaatgttttgtatattatatgttgagatttttcagatcttttagcccagtcatatgttctcacgtctcctactttttgtttctcgtataaaactttttgatttgaaccaaaacagtgaagatgacaacacacaccaattggatgataattgtagcgggggcggtgggggggagaagtccacgccatccatcatgcaaaattctttcagatgaggtgggaaggccgtgtacatagttatgtaaaaagagattgcttcatgagctaatggttcatatatgcaaaagggtaagatgaaagctttactttgtacaaatgtaaatagataaagtaacataatacattaatacttcttaaaatgtgctatttgcaaacttacttaatatcagtgaacacagtcggctaaagctgtgttcccatatattgttatagacagctaaacccttcaactatgcaatgaatgttcgggcttttcacaaaagcccgcctaactcaaaggagccttttcaaatccatttacagcatacttaaggtcatattttccctgaacaagcgcttacgtgatatgactctgttttccttgcttgttttttttcaaacggagaaacatcctgttttgcaaattggaccccaggctggaacttagcatctgaagttgccgcttgtgggctctgggggaaagtgtagccccggagaggtaactgaggacatgagcaaccagtgccagggagggtgggatttgccagatgccaaaatcaggggacgggtggtggtgtctgtcagacacacacaggtcgccagtgacttcacacacacctcatgtgagaaccatgccttttttagtgtgtcctatttcatacctgtacacacttcctcgttttgtaatgagatttacttacacccaaacagatcctgaaagaaagcttcaagttttctcagatgatggatatgttttcactgtattcaataactgacggatgtaaggtgcacgtttcctgatgtgacgcactgtattccagctggtgatcaagtctgggaacagccgtaacaggtcaaccttgtggagccatcgcgagttagagggtgaaagatggcagaaaaaaaagtcttgtgtgtgagtgtgttttttgagtttgcatcaatcttaatgtctcttcataatacttttataatacattaagcctcttgtctacatatttggagagaatatgactttactagcagagaaatacaatatatcttgtctactggactgtaaaatatatgtatgaaataaaattagttccatttggtcttctagtatattaaagtgctatctgacgttgttatcctgtttttgcaaaaaaaaaaaaaaaaaa

SEQ ID NO: 6 - protein, 1235 aa, Homo sapiens
MDGPTRGHGLRKKRRSRSQRDRERRSRGGLGAGAAGGGGAGRTRALSLASSSGSDKEDNGKPPSSAPSRPRPPRRKRRESTSAEEDIIDGFAMTSFVTFEALEKDVALKPQERVEKRQTPLTKKKREALTNGLSFHSKKSRLSHPHHYSSDRENDRNLCQHLGKRKKMPKALRQLKPGQNSCRDSDSESASGESKGFHRSSSRERLSDSSAPSSLGTGYFCDSDSDQEEKASDASSEKLFNTVIVNKDPELGVGTLPEHDSQDAGPIVPKISGLERSQEKSQDCCKEPIFEPVVLKDPCPQVAQPIPQPQTEPQLRAPSPDPDLVQRTEAPPQPPPLSTQPPQGPPEAQLQPAPQPQVQRPPRPQSPTQLLHQNLPPVQAHPSAQSLSQPLSAYNSSSLSLNSLSSSRSSTPAKTQPAPPHISHHPSASPFPLSLPNHSPLHSFTPTLQPPAHSHHPNMFAPPTALPPPPPLTSGSLQVAGHPAGSTYSEQDILRQELNTRFLASQSADRGASLGPPPYLRTEFHQHQHQHQHTHQHTHQHTFTPFPHAIPPTAIMPTPAPPMFDKYPTKVDPFYRHSLFHSYPPAVSGIPPMIPPTGPFGSLQGAFQPKLTDPFRPMLRKPGKWCAMHVHIAWQIYHHQQKVKKQMQSDPHKLDFGLKPEFLSRPPGPSLFGAIHHPHDLARPSTLFSAAGAAHPTGTPFGPPPHHSNFLNPAAHLEPFNRPSTFTGLAAVGGNAFGGLGNPSVTPNSMFGHKDGPSVQNFSNPHEPWNRLHRTPPSFPTPPPWLKPGELERSASAAAHDRDRDVDKRDSSVSKDDKERESVEKRHSSHPSPAPVLPVNALGHTRSSTEQIRAHLNTEAREKDKPKERERDHSESRKDLAADEHKAKEGHLPEKDGHGHEGRAAGEEAKQLARVPSPYVRTPVVESARPNSTSSREAEPRKGEPAYENPKKSSEVKVKEERKEDHDLPPEAPQTHRASEPPPPNSSSSVHPGPLASMPMTVGVTGIHPMNSISSLDRTRMMTPFMGISPLPGGERFPYPSFHWDPIRDPLRDPYRELDIHRRDPLGRDFLLRNDPLHRLSTPRLYEADRSFRDREPHDYSHHHHHHHHPLSVDPRREHERGGHLDERERLHMLREDYEHTRLHSVHPASLDGHLPHPSLITPGLPSMHYPRISPTAGNQNGLLNKTPPTAALSAPPPLISTLGGRPVSPRRTTPLSAEIRERPPSHTLKDIEAR

SEQ ID NO: 7 - DNA, 1678 bp, Homo sapiens
gggagctgcgctcgcagtttcgccctctcttccgctaatgattgcattattatgctcccctctctggggggtctcgcccctcttgggtcgctccggagccccggcctcccctggctgcatttcttaaaaatttgggagcctgggagtgagttttctccgaggcgtgtgtgagaggcggcgggggtgttttcctgcgcgaggggcgggtgaagttcattgcccccacttttcccgcgacctttttcggacccgattttggatcgagttgaggggggcgcgggcgttttcggggggcggggggcgcggcggagaatggccgcggggagggctccccggagcctcccagtctcttgatcaaagcattccgctattctgatttattgcttgcttggtgagttatttttttttcctctaaaggagacctgtgtgttcagccattactttgctcggcgctgctcccaggcatctccgaccctcggtgctgtggggagccccacacttgggctcctcgcctctcgccctcgctccccgtccctcctcccctctctccgccccttcccccttttctttctcctctctttcttcccctctctcccttctttcggccgccgtctcccccgcgccctcctcggggcggagggaagccgtgaagggggagggagggctcggtgtcaatttttttttgtgtggctgcggccgtagcctgtggcgggcaagcggggagaccccggcgcagcagaaccatggatggcccgacgcggggccatggactccgcaaaaagcggcggtcgcggtcgcagcgagaccgggagaggcgctcccggggcgggctgggggccggcgcggccggcggcggcggggctggccggacccgggcgctctcactcgcctcgtcgtcgggctccgacaaggaagacaatgggaagcccccgtcctccgccccgtcccggcccagacccccgcggaggaagcggagagagtccacctcggcagaagaggacatcattgatggatttgccatgaccagctttgtcacttttgaagcgctggagaaagatgtagcacttaagcctcaggaacgtgtggagaaacgccagacgcccctgaccaagaagaaacgagaagcacttaccaatggcttgtcctttcattcaaagaagagcagactcagccacccacaccactacagctcagatcgagaaaatgaccgcaatctctgccagcaccttgggaagagaaagaaaatgccgaaggcactcagacagctcaagccaggacagaacagctgcagggacagtgacagtgaaagtgccagtggagaatccaagggcttccaccggagcagctctcgggaaaggctcagtgatagttcagctccttccagcttgggaacaggctacttcagatcagggaagatgtgccttggagaggaagcatgtcttaaatctggaaatgatatgaagagggatgtcagcaacacttcatcctgggccagtaatagggagagtttcttttctctcgtcaaattgcttaaaggattctagttccgtttggtgtggtcactcacatttgaattctaatactctatgtgatatagattctgttgactactgttagcgtgaccccaatgagaaattaaacacttccctccttttcaaaaaaaaaaaaaaaaaaaaaa

SEQ ID NO: 8 - protein, 266 aa, Homo sapiens
MDGPTRGHGLRKKRRSRSQRDRERRSRGGLGAGAAGGGGAGRTRALSLASSSGSDKEDNGKPPSSAPSRPRPPRRKRRESTSAEEDIIDGFAMTSFVTFEALEKDVALKPQERVEKRQTPLTKKKREALTNGLSFHSKKSRLSHPHHYSSDRENDRNLCQHLGKRKKMPKALRQLKPGQNSCRDSDSESASGESKGFHRSSSRERLSDSSAPSSLGTGYFRSGKMCLGEEACLKSGNDMKRDVSNTSSWASNRESFFSLVKLLKGF

SEQ ID NO: 9 - DNA, 22 bp, Artificial
  Chemically synthesized
cacacagtgcaagaggcaatac

SEQ ID NO: 10 - DNA, 22 bp, Artificial
  Chemically synthesized
gatgcacttcggagttgatacc

SEQ ID NO: 11 - DNA, 23 bp, Artificial
  Chemically synthesized
ttaaccaacacataccaatcgtt

SEQ ID NO: 12 - DNA, 22 bp, Artificial
  Chemically synthesized
gatttctggtgtctgccaacat

SEQ ID NO: 13 - DNA, 22 bp, Artificial
  Chemically synthesized
gaaatagagcactgccaagacc

SEQ ID NO: 14 - DNA, 24 bp, Artificial
  Chemically synthesized
cattggatagaaattacagcctga

SEQ ID NO: 15 - DNA, 22 bp, Artificial
  Chemically synthesized
accattggatgacatttgtgtt

SEQ ID NO: 16 - DNA, 25 bp, Artificial
  Chemically synthesized
ggtagtttattgtcagagaaagcaa

SEQ ID NO: 17 - DNA, 23 bp, Artificial
  Chemically synthesized
catttattctttgcagacacctg

SEQ ID NO: 18 - DNA, 25 bp, Artificial
  Chemically synthesized
tttaaagaattgagcaacatgaaca

SEQ ID NO: 19 - DNA, 22 bp, Artificial
  Chemically synthesized
tatcccaggttaactcgaatgg

SEQ ID NO: 20 - DNA, 25 bp, Artificial
  Chemically synthesized
tcaggtttttaaaattgtcagtgtc

SEQ ID NO: 21 - DNA, 23 bp, Artificial
  Chemically synthesized
attttggaggcagaatgctataa

SEQ ID NO: 22 - DNA, 23 bp, Artificial
  Chemically synthesized
ttttgcccaaacacaaatatgat

SEQ ID NO: 23 - DNA, 22 bp, Artificial
  Chemically synthesized
aggctgtgcttcaaaacttgta

SEQ ID NO: 24 - DNA, 22 bp, Artificial
  Chemically synthesized
gtaacaccagcaaaaccaaaca

SEQ ID NO: 25 - DNA, 23 bp, Artificial
  Chemically synthesized
aaatcgtgatttgttgattttgg

SEQ ID NO: 26 - DNA, 24 bp, Artificial
  Chemically synthesized
tttttgttttgctcagtggaatta

SEQ ID NO: 27 - DNA, 22 bp, Artificial
  Chemically synthesized
gtagttggatgtgatggctgtg

SEQ ID NO: 28 - DNA, 24 bp, Artificial
  Chemically synthesized
tggtaatttccaccttacctgttt

SEQ ID NO: 29 - DNA, 22 bp, Artificial
  Chemically synthesized
atatattgcccagacagcttgg

SEQ ID NO: 30 - DNA, 22 bp, Artificial
  Chemically synthesized
ttggtttttcagattcgagtga

SEQ ID NO: 31 - DNA, 22 bp, Artificial
  Chemically synthesized
ggtttgctagcattgcaatatg

SEQ ID NO: 32 - DNA, 22 bp, Artificial
  Chemically synthesized
gaaacaaaccattggtggaact

SEQ ID NO: 33 - DNA, 23 bp, Artificial
  Chemically synthesized
aacactgttctacaccagctcag

SEQ ID NO: 34 - DNA, 22 bp, Artificial
  Chemically synthesized
tcttagcttcattccccagaaa

SEQ ID NO: 35 - DNA, 22 bp, Artificial
  Chemically synthesized
tcagagtattcctggggaagtg

SEQ ID NO: 36 - DNA, 22 bp, Artificial
  Chemically synthesized
tttgtcagttgggttagttcca

SEQ ID NO: 37 - DNA, 22 bp, Artificial
  Chemically synthesized
tgctatgagaccacctatggaa

SEQ ID NO: 38 - DNA, 22 bp, Artificial
  Chemically synthesized
agtctgattgcaggcatcttct

SEQ ID NO: 39 - DNA, 22 bp, Artificial
  Chemically synthesized
gaggatttggtccaatgttgtt

SEQ ID NO: 40 - DNA, 22 bp, Artificial
  Chemically synthesized
ggcttgtgtgtccacctctagt

SEQ ID NO: 41 - DNA, 22 bp, Artificial
  Chemically synthesized
attttgccatcgacctttgtag

SEQ ID NO: 42 - DNA, 23 bp, Artificial
  Chemically synthesized
tgtgcaggctcttaaaaatcaac

SEQ ID NO: 43 - DNA, 24 bp, Artificial
  Chemically synthesized
ctatgcagtgtcatctcctaccac

SEQ ID NO: 44 - DNA, 24 bp, Artificial
  Chemically synthesized
ttggaaaattcctacctaagttga

SEQ ID NO: 45 - DNA, 22 bp, Artificial
  Chemically synthesized
acttactcagatgcccttcctg

SEQ ID NO: 46 - DNA, 23 bp, Artificial
  Chemically synthesized
tggcaagttgttttcctgatatt

SEQ ID NO: 47 - DNA, 22 bp, Artificial
  Chemically synthesized
gacatcaagggagggagtaaag

SEQ ID NO: 48 - DNA, 22 bp, Artificial
  Chemically synthesized
ctatcccctcaaaacaaaacca

SEQ ID NO: 49 - DNA, 24 bp, Artificial
  Chemically synthesized
ggtgttttagagtcagtgctgatg

SEQ ID NO: 50 - DNA, 24 bp, Artificial
  Chemically synthesized
agaacaaccacgtaactttcctgt

SEQ ID NO: 51 - DNA, 22 bp, Artificial
  Chemically synthesized
tgcagccctaaatcttatcgac

SEQ ID NO: 52 - DNA, 22 bp, Artificial
  Chemically synthesized
cctgagaactccgtactcacaa

SEQ ID NO: 53 - DNA, 22 bp, Artificial
  Chemically synthesized
ctgttgtgattcttgtgggaga

SEQ ID NO: 54 - DNA, 25 bp, Artificial
  Chemically synthesized
cagcaaaatgaataatgtaaaaacc

SEQ ID NO: 55 - DNA, 22 bp, Artificial
  Chemically synthesized
ctgacggagctgtagtgaagtg

SEQ ID NO: 56 - DNA, 23 bp, Artificial
  Chemically synthesized
cacgggtctttagaacacctcta

(19) United States
(12) Patent Application Publication
(10) Pub. No.: US 2011/0118135 A1
(43) Pub. Date: May 19, 2011

(54) Mutations in Contaction Associated Protein 2 (CNTNAP2) are Associated with Increased Risk for Ideopathic Autism

(76) Inventors: Matthew W. State, Branford, CT (US); Brian J. O'roark, New Haven, CT (US); Richard P. Lifton, North Haven, CT (US)

(21) Appl. No.: 12/811,994

(22) PCT Filed: Jan. 9, 2009
(86) PCT No.: PCT/US09/30620

§ 371 Date: Dec. 29, 2010

(51) Int. Cl. C40B 30/04; C12Q 1/68; G01N 33/68

(52) U.S. Cl. 506/9; 435/6; 435/7.1; 436/501; 435/7.92

(57) ABSTRACT

The present invention provides compositions and methods for the examination of cells, tissues, and fluids, collectively known as body samples, to identify human subjects at-risk of developing Autism Spectrum Disorder by detecting a chromosomal abnormality or variant in the CNTNAP2 gene, the AUTS2 gene, or both.

### CROSS-REFERENCE TO RELATED APPLICATIONS

This application is the U.S. national phase application filed under 35 U.S.C. §371 claiming benefit to International Patent Application No. PCT/US2009/030620, filed on Jan. 9, 2009, which is entitled to priority under 35 U.S.C. §119(a) to U.S. Provisional Patent Application No. 61/010,676, filed on Jan. 9, 2008, each of which application is hereby incorporated herein by reference in its entirety.

### BACKGROUND OF THE INVENTION

Autism spectrum disorders (ASD) are a group of related neurodevelopmental syndromes of complex genetic etiology (Gupta and State, 2007, Biol. Psychiatry 61:429-437). The diagnostic criteria for autism in general include qualitative impairment in social interaction, as manifest by impairment in the use of nonverbal behaviors such as eye-to-eye gaze, facial expression, body postures, and gestures, failure to develop appropriate peer relationships, and lack of social sharing or reciprocity. Patients may have impairments in communication, such as a delay in, or total lack of, the development of spoken language. In patients who do develop adequate speech, there may remain a marked impairment in the ability to initiate or sustain a conversation, as well as stereotyped or idiosyncratic use of language. Patients may also exhibit restricted, repetitive and stereotyped patterns of behavior, interests, and activities, including abnormal preoccupation with certain activities and inflexible adherence to routines or rituals. Fundamental impairment in some but not all of these domains defines a spectrum of conditions that includes Asperger syndrome and Pervasive Developmental Disorder Not Otherwise Specified (PDD-NOS). In the DSM-IV, rare developmental disorders including Rett Syndrome and Childhood Disintegrative Disorder (Tuchman et al., 2002, Lancet Neurol. 1:352-358) are grouped in the same diagnostic category. A majority of patients with ASD have mental retardation (MR) in addition to their social disability and up to one-third suffer from seizures (Tuchman et al., 2002, Lancet Neurol. 1:352-358). Individuals with ASD also show an increased burden of chromosomal abnormalities (Gupta and State, 2007, Biol. Psychiatry 61:429-437) and de novo rare copy number variants (Sebat et al., 2007, Science 316:445-449).

Despite multiple lines of evidence suggesting a complex genetic etiology, common ASD variants have been extremely difficult to identify (Gupta and State, 2007, Biol. Psychiatry 61:429-437). In addition, to date there has not been a convergence between the rare mutations identified in nonsyndromic autism, such as those in the Neuroligin gene family (Jamain et al., 2003, Nature Genetics 34:27-29; Laumonnier et al., 2004, Am. J. Hum. Genet. 74:552-557; Vincent et al., 2004, Am. J. Med. Genet. B. Neuropsychiatr. Genet. 129:82-84; Gauthier et al., 2005, Am. J. Med. Genet. B. Neuropsychiatr. Genet. 132:74-75; Ylisaukko-oja et al., 2005, Eur. J. Hum. Genet. 13:1285-1292; Blasi et al., 2006, Am. J. Med. Genet. 13:1285-1292), and those genomic regions most strongly implicated by nonparametric linkage or common variant association studies. Difficulties in clarifying the genetic substrates of ASD likely reflect the combination of marked locus and allelic heterogeneity, the absence of reliable biological diagnostic markers, and the likelihood that any contributing common alleles will be found to carry quite small increments of risk, requiring very large sample sizes to definitively confirm their contributions (Gupta and State, 2007, Biol. Psychiatry 61:429-437).

There is a long-standing need in the art to identify specific chromosomal abnormalities or genetic variants that contribute to the pathophysiology of ASD. The present invention meets this need.

### SUMMARY OF THE INVENTION

In one embodiment the invention includes a method of identifying a human subject at-risk of developing Autism Spectrum Disorder (ASD), the method comprising obtaining a body sample from the subject; detecting at least one chromosomal abnormality in a gene selected from the group consisting of the CNTNAP2 gene, the AUTS2 gene, and combinations thereof, where if at least one chromosomal abnormality is detected in the gene, then the subject is at-risk of developing ASD. In one aspect, the subject is selected from the group consisting of a fetus, a neonate, and a child. In another aspect, the child is less than or equal to 5 years old. In another aspect, the body sample is selected from the group consisting of a tissue, a cell, and a bodily fluid. In still another aspect, the assay is selected from the group consisting of a PCR assay, a sequencing assay, an assay using a probe array, an assay using a gene chip, and an assay using a microarray.

In another embodiment, the invention includes a method of identifying a human subject at-risk of developing Autism Spectrum Disorder (ASD), the method comprising: obtaining a body sample from the subject; detecting at least one disrupted transcription of a gene selected from the group consisting of the CNTNAP2 gene, the AUTS2 gene, and combinations thereof, where if at least one disrupted transcript is detected in the gene, then the subject is at-risk of developing ASD. In one aspect, the method comprises an assay for mRNA selected from the group consisting of CNTNAP2 mRNA, AUTS2 mRNA, or a combination thereof. IN another aspect, the assay comprises Northern blot analysis, in situ hybridization, or RT-PCR. In still another aspect, the method comprises an assay for CNTNAP2 protein, AUTS2 protein, or a combination thereof. In yet another aspect, the assay comprises a Western blot analysis, radioimmunoassay (RIA), and immunoassay, chemiluminescent assay, or enzyme-linked immunosorbent assay (ELISA). In still another aspect, the subject is selected from the group consisting of a fetus, a neonate, and a child. In yet another aspect, the child is less than or equal to 5 years old. In another aspect, the body sample is selected from the group consisting of a tissue, a cell, and a bodily fluid.

In still another embodiment the present invention includes a method for determining in a human subject, the presence or absence of a sequence variation in a gene selected from the group consisting of CNTNAP2, AUTS2, or a combination thereof, the method comprising obtaining a body sample from the subject; detecting at least one sequence variation in a gene selected from the group consisting of the CNTNAP2 gene, the AUTS2 gene, and combinations thereof, wherein if at least one sequence variation is detected in either of the genes, then the subject is at-risk of developing ASD. In one aspect, the subject is selected from the group consisting of a fetus, a neonate, and a child. In another aspect, the child is less than or equal to 5 years old. In yet another aspect, the body sample is selected from the group consisting of a tissue, a cell, and a bodily fluid. In still another aspect, the assay is selected from the group consisting of a PCR assay, a sequencing assay, an assay using a probe array, an assay using a gene chip, and an assay using a microarray. In another aspect, the sequence variation in said CNTNAP2 gene is selected from the group consisting of I869T, R1119H, D1129H, I1253T, I1278I, T218M, L226M, R283c, S382N, E680K, W134G, L292Q, V708A, Q921R, R1027T, and V1157A.

In still another embodiment, the invention includes a method of identifying a human subject at-risk of germ-line transmission of Autism Spectrum Disorder (ASD) to progeny of the subject, the method comprising: obtaining a body sample from the subject; detecting at least one sequence variation of a gene selected from the group consisting of the CNTNAP2 gene, the AUTS2 gene, and combinations thereof, wherein if at least one sequence variation is detected in the gene, then the subject is at-risk of transmitting ASD to the progeny. In one aspect, the method comprises an assay for mRNA selected from the group consisting of CNTNAP2 mRNA, AUTS2 mRNA, or a combination thereof. In another aspect, the assay comprises Northern blot analysis, in situ hybridization, or RT-PCR. In still another aspect, the method comprises an assay for CNTNAP2 protein, AUTS2 protein, or a combination thereof. In yet another aspect, the assay comprises a Western blot analysis, radioimmunoassay (MA), and immunoassay, chemiluminescent assay, or enzyme-linked immunosorbent assay (ELISA). In another aspect, the body sample is selected from the group consisting of a tissue, a cell, and a bodily fluid. In yet another aspect, the sequence variation in said CNTNAP2 gene is selected from the group consisting of I869T, R1119H, D1129H, I1253T, I1278I, T218M, L226M, R283c, S382N, E680K, W134G, L292Q, V708A, Q921R, R1027T, and V1157A.

In yet another embodiment, the invention includes a method of prenatally identifying a human subject at-risk of germ-line transmission of Autism Spectrum Disorder (ASD) to progeny of the subject, the method comprising: obtaining a body sample from the subject; detecting at least one sequence variation of a gene selected from the group consisting of the CNTNAP2 gene, the AUTS2 gene, and combinations thereof, wherein if at least one sequence variation is detected in the gene, then the subject is at-risk of transmitting ASD to the progeny. In one aspect, the method comprises an assay for mRNA selected from the group consisting of CNTNAP2 mRNA, AUTS2 mRNA, or a combination thereof. In another aspect, the assay comprises Northern blot analysis, in situ hybridization, or RT-PCR. In still another aspect, the method comprises an assay for CNTNAP2 protein, AUTS2 protein, or a combination thereof. In yet another aspect, the assay comprises a Western blot analysis, radioimmunoassay (RIA), and immunoassay, chemiluminescent assay, or enzyme-linked immunosorbent assay (ELISA). IN another aspect, the body sample is selected from the group consisting of a tissue, a cell, and a bodily fluid. In still another aspect, the sequence variation in said CNTNAP2 gene is selected from the group consisting of I869T, R1119H, D1129H, I1253T, I1278I, T218M, L226M, R283C, S382N, E680K, W134G, L292Q, V708A, Q921R, R1027T, and V1157A.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1D, is a series of images depicting mapping of a de novo inversion (inv(7)(q11.22;q35)) in a child with developmental delay.

FIG. 1A is a diagram depicting the pedigree of a family with an affected male child with developmental delay. The parents, grandparents, and two older siblings are not affected with a neurodevelopmental disorder. FIG. 1B is an image depicting G-banded metaphase chromosomes. Ideogram for normal (left) and inverted (right) chromosomes are presented. FIG. 1C depicts FISH mapping of q35 breakpoints. The image shows the two bacterial artificial chromosomes (BACs) that span the breaks. The experimental probe is seen at the expected positions on the normal (nml) chromosome 7q35. Two fluorescence signals are visible on the inverted (inv) chromosomes indicating that the probes span the break points. Photographs were taken with a 100× objective lens. FIG. 1D depict FISH mapping of q35 q11.22 breakpoints. The image shows the two bacterial artificial chromosomes (BACs) that span the breaks. The experimental probe is seen at the expected positions on the normal (nml) chromosome 7q11.22. Two fluorescence signals are visible on the inverted (inv) chromosomes indicating that the probes span the break points. Photographs were taken with a 100× objective lens. FIG. 1E is a schematic diagram depicting the location of the spanning BACs relative to the disrupted CNTNAP2 gene. FIG. 1E shows that the edges of the BAC RP11-1012D24 are 1314 kb and 821 kb away from the centromeric and telomeric ends of CNTNAP2. FIG. 1F is a schematic diagrams depicting the location of the spanning BACs relative to the disrupted AUTS2 gene. FIG. 1F shows that the edges of the BAC RP11-709J20 are 926 kb and 110 kb away from the centromeric and telomeric ends of AUTS2.

FIG. 2, comprising FIG. 2A through FIG. 2F, depicts a series of images depicting expression of Cntnap2 mRNA in postnatal mouse brain. All panels represent coronal sections and are shown in anterior to posterior order. Ctx, cortex; CPu, caudate putamen; Se, septum; GP, globus pallidus; Th, thalamus; Hip; hippocampal formation; A, amygdala; HTh, hypothalamus; SC, superior colliculus; PAG, periaqueductal gray; Pn, pontine nuclei.

FIG. 3, comprising FIG. 3A through FIG. 3D, is a series of images depicting expression and biochemical analyses of CNTNAP2/Cntnap2. FIG. 3A is a photomicrograph depicting CNTNAP2/Cntnap2 expression in human temporal cortex (6 years of age). Cortical layers are designated II, III, IV, and V. FIG. 3B is a photomicrograph depicting CNTNAP2/Cntnap2 expression in human temporal cortex (58 years of age). Cortical layers are designated II, III, IV, and V. FIG. 3C is a photomicrograph depicting CNTNAP2/Cntnap2 expression in mouse neocortex (postnatal day 7). Cortical layers are designated II/III, IV, V, and VI. FIG. 3D is an image depicting co-fractionation of Cntn2/TAG-1 and Cntnap2 in synaptic plasma membranes obtained from rat forebrain homogenate (homog.) subfractionated into postnuclear supernatant (S1), synaptosomal supernatant (S2), crude synaptosomes (P2), synaptosomal membranes (LP1), crude synaptic vesicles (LP2), synaptic plasma membranes (SPM), and mitochondria (mito.). The synaptic membrane protein N-cadherin and the synaptic vesicle protein synaptotagmin 1 served as markers for these respective fractions. Numbers on the left indicate positions of molecular weight markers.

FIG. 4, comprising FIG. 4A and FIG. 4B, is a series of images depicting the identification of rare unique nonsynonymous variants in the CNTNAP2 protein. FIG. 4A is a diagram depicting the CNTNAP2 protein and highlighting the location of unique predicted deleterious variants (modified from SMART). The locations of patient variants are indicated. Variants G7315, I869T, R1119H, D1129H, I1253T, and T12781 are predicted by the use of bioinformatics tools to be deleterious or are located at conserved sites. Asterisk indicates variant was identified in three independent families; SP, signal peptide; FA58C, coagulation factor 5/8 C-terminal domain; LamG, Laminin G domain; EGF and EFG-L, epidermal growth factor-like domains; TM, transmembrane domain; 4.1M, putative band 4.1 homologs' binding motif; black vertical bar, C-terminal type II PDZ binding sequence. Figure is to scale. FIG. 4B is an image depicting pedigrees for all families with variants predicted to be deleterious at conserved sites (I to XIII) or which all affected relatives carry the identified variant (IX-X). The individuals carrying the suspect allele are noted and are heterozygous. The brothers inheriting the D1129H variant are monozygotic twins. Affected status was calculated with the AGRE diagnosis algorithm, which is based on ADI-R scores. Blackened symbols represent an autism diagnosis, half-filled symbols indicate a not-quite-autism (NQA) diagnosis, and crosshatched individuals have a broad spectrum diagnosis.

FIG. 5 is an image depicting a ClustalW alignment of top BlastP hits to CNTNAP2. Unique variants identified in the case (N407S; N418D; Y716C; G731S; I869T; R906H; R1119H; D1129H; A1227T; I1253T; T12781) and control groups (R114Q; T218M; L226M; R283c; S382N; E680K; P699Q; G779D; D1038N; V1102A; S1114G). Amino acids marked with gray are identical to human sequence. The following fall into the same broad physio-chemical group: T218S; L226F; N418G; Y716H or S; G779S; I869L; D1038E; V1102I or L; S1114N; A1227V; I1253P; and T127H. An asterisk (*) identifies residues or nucleotides that are identical in all sequences in the alignment. A colon (:) designates conserved substitutions. A period (.) denotes semiconserved substitutions. Homo sapiens, NP_{—}054860.1; Pan troglodytes, XP_{—}519462.2; Macaca mulatta, XP_{—}001094652.1; Pongo pygmaeus, Q5RD64; Mus musculus, NP_{—}001004357.1; Monodelphis domestica, XP_{—}001368218.1; Ornithorhynchus anatinus, XP_{—}001505555.1; Xenopus tropicalis, NP_{—}001072732.1; Danio rerio, XP_{—}691801.2; Tetraodon nigroviridis, CAG11627.1.

### DETAILED DESCRIPTION OF THE INVENTION

The present invention provides compositions and methods for the examination of cells, tissues, and fluids, collectively known as body samples, to identify human subjects at-risk of developing Autism Spectrum Disorder.

The method of the invention comprises a method of detecting at least one chromosomal abnormality or sequence variation in the CNTNAP2 gene, the AUTS2 gene, or both, in a body sample collected from a human subject. Chromosomal abnormalities include, but are not limited to, chromosomal deletions, duplications, inversions, insertions, and translocations. Sequence variations include, but are not limited to, unique non-synonymous variants or alleles.

In another embodiment, the invention comprises the method of detecting a disrupted CNTNAP2 transcript, a disrupted AUTS2 transcript, or a combination thereof, wherein said transcript may be detected at either the mRNA or protein level.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The phrase “body sample” as used herein, is intended any sample comprising a cell, a tissue, or a bodily fluid in which expression of a CNTNAP2 or AUTS2 gene or gene product can be detected. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

The phrase “at-risk” as used herein refers to a subject with a greater than average likelihood of developing Autism Spectrum Disorder.

As used herein, an “allele” is one of several alternate forms of a gene or non-coding regions of DNA that occupy the same position on a chromosome.

A “biomarker” of the invention is any detectable chromosomal abnormality contributes to a subject being at-risk for ASD. The chromosomal abnormality may be detected at either the nucleic acid or protein level.

The term “child”, as used herein, refers to a human subject between the ages of 0 and 18 years of age, including neonates.

The term “chromosomal abnormality,” as used herein, refers to a deviation between the structure of the subject chromosome and a normal homologous chromosome. The term “normal” refers to the predominate karyotype banding pattern or a nucleic acid sequence found in healthy individuals of a particular species. A chromosomal abnormality can be numerical or structural, and includes, but is not limited to, aneuploidy, polyploidy, inversion, trisomy, monosomy, chromosomal deletions, duplications, inversions, insertions, and translocations. A chromosomal abnormality of the invention is correlated with an increased risk of developing ASD.

A “sequence variation,” as used herein, refers to a unique nonsynonomous variant or allele of a subject's gene from a normal homologous gene. A sequence variation of the invention is correlated with an increased risk of developing ASD. As defined herein, a single nucleotide polymorphism (“SNP”) is not a chromosomal abnormality.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Substantially complementary to” refers to probe or primer sequences which hybridize to the sequences listed under stringent conditions and/or sequences having sufficient homology with test polynucleotide sequences, such that the allele specific oligonucleotide probe or primers hybridize to the test polynucleotide sequences to which they are complimentary.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Polymorphism” as used herein refers to a sequence variation in a gene which is not necessarily associated with pathology.

“Mutation” as used herein refers to an altered genetic sequence which results in the gene coding for a non-functioning protein or a protein with reduced or altered function. Generally, a deleterious mutation is associated with pathology or the potential for pathology.

“Allele specific detection assay” as used herein refers to an assay to detect the presence or absence of a predetermined sequence variation in a test polynucleotide or oligonucleotide by annealing the test polynucleotide or oligonucleotide with a polynucleotide or oligonucleotide of predetermined sequence such that differential DNA sequence based techniques or DNA amplification methods discriminate between normal and mutant.

“Sequence variation locating assay” as used herein refers to an assay that detects a sequence variation in a test polynucleotide or oligonucleotide and localizes the position of the sequence variation to a subregion of the test polynucleotide, without necessarily determining the precise base change or position of the sequence variation.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

As used herein, the term “fragment,” as applied to a protein or peptide, refers to a subsequence of a larger protein or peptide. A “fragment” of a protein or peptide can be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

By the term “specifically binds,” as used herein, is meant an antibody which recognizes and binds a biomarker or fragment thereof, but does not substantially recognize or bind other molecules in a sample.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

### Description

The present invention provides compositions and methods for identifying a human subject at-risk of developing Autism Spectrum Disorder (ASD). In one embodiment, the present invention comprises a method for identifying a human subject at-risk of developing ASD, where the method comprises detecting at least one chromosomal abnormality or sequence variation that contributes to the etiology of cognitive and social delays associated with ASD, wherein if at least one such chromosomal abnormality or sequence variation is detected, then said subject is at-risk of developing ASD.

In another embodiment, the present invention comprises a method for identifying a human subject at-risk of developing ASD where the method comprises detecting at least one disrupted gene product, including an mRNA and/or protein, that contributes to the etiology of cognitive, behavioral, language, or social delays associated with ASD. A disrupted gene product of the invention comprises any gene product that is a variant or mutant of a normal gene product and cannot fulfill the normal gene product's function, and thus, contributes to the etiology of ASD. If at least one such disrupted gene product is detected according to the method of the invention, then the subject is at-risk of developing ASD.

In still another embodiment, the invention comprises a method of detecting the presence or absence of at least one sequence variant in a gene that contributes to the etiology of cognitive, behavioral, language, or social delays associated with ASD, wherein when the presence of at least one such sequence variant is detected, then the subject is at-risk of developing ASD.

In a preferred embodiment, the present invention identifies an abnormality or sequence variation in the CNTNAP2 gene, the AUTS2 gene, or a combination thereof, as contributing to the etiology of cognitive, behavioral, language, or social delays associated with ASD. Accordingly, an abnormality or sequence variation in the CNTNAP2 gene, the AUTS2 gene, or combinations thereof, is identified herein as a biomarker for a subject at-risk of developing ASD. In another embodiment, the present invention identifies a disrupted product of the CNTNAP2 gene, the AUTS2 gene, or a combination thereof as a biomarker for a subject at-risk of developing ASD.

In one embodiment, the present invention comprises a method for identifying a human subject at-risk of developing ASD, where the method comprises detecting at least one chromosomal abnormality or sequence variation in the CNTNAP2 gene, the AUTS2 gene, or combinations thereof that contributes to the etiology of cognitive, behavioral, language, or social delays associated with ASD, wherein if at least one chromosomal abnormality or sequence variation in the CNTNAP2 gene, the AUTS2 gene, or combinations thereof is detected, then said subject is at-risk of developing ASD.

In another embodiment, the present invention comprises a method for identifying a human subject at-risk of developing ASD where the method comprises detecting at least one disrupted gene product of the CNTNAP2 gene, the AUTS2 gene, or combinations thereof; including an mRNA and/or protein that contributes to the etiology of cognitive, behavioral, language, or social delays associated with ASD. If at least one disrupted gene product of the CNTNAP2 gene, the AUTS2 gene, or combinations thereof is detected, then the subject is at-risk of developing ASD.

In still another embodiment, the invention comprises a method of detecting the presence or absence of at least one sequence variant in the CNTNAP2 gene, the AUTS2 gene, or combinations thereof that contributes to the etiology of cognitive, behavioral, language, or social delays associated with ASD, wherein when the presence of at least one sequence variant in the CNTNAP2 gene, the AUTS2 gene, or combinations thereof is detected, then the subject is at-risk of developing ASD.

The CNTNAP2 gene maps to a 2.3 MB genomic region on 7q35 and encodes a member of the neurexin family which functions as cell adhesion molecules and receptors. The nucleic acid sequence corresponds to the sequence deposited in National Center for Biotechnology Information (NCBI) as NM_{—}014141 (SEQ ID NO: 1) and encodes the protein that corresponds to NCBI sequence NP_{—}054860 (SEQ ID NO: 2).

A sequence variation of the CNTNAP2 gene comprises any amino acid substitution that is predicted to have a deleterious effect on the affected individual in terms of contributing to the etiology of cognitive, behavioral, language, or social delays associated with ASD. Examples of such sequence variations include, but are not limited to, I869T, R1119H, D1129H, I1253T, I1278I, T218M, L226M, R283c, S382N, E680K, W134G, L292Q, V708A, Q921R, R1027T, and V1157A.

The AUTS2 gene maps to a 1.2 MB genomic region of 7q11.22 and is known to have several isoforms. AUTS2 isoform one corresponds to the nucleic acid sequence NM_{—}015570.2 (SEQ ID NO: 3) which encodes NP_{—}056385.1 (SEQ ID NO: 4). AUTS2 isoform 2 corresponds to the nucleic acid sequence NM_{—}001127231.1 (SEQ ID NO: 5) which encodes NP_{—}001120703.1 (SEQ ID NO: 6). AUTS2 isoform 3 corresponds to the nucleic acid sequence NM_{—}001127232.1 (SEQ ID NO: 7) which encodes NP_{—}001120704.1 (SEQ ID NO: 8).

Any method available in the art for detecting a chromosomal abnormality, sequence variation, or a disrupted gene product is encompassed herein. The invention should not be limited to those methods for detecting chromosomal abnormalities, sequence variations, or disrupted gene products recited herein, but rather should encompasses all known or heretofore unknown methods for detection as are, or become, known in the art.

Methods for detecting a chromosomal abnormality, sequence variation, or disrupted gene transcription of CNTNAP2 and AUTS2 comprise any method that interrogates the CNTNAP2 or AUTS2 gene or their products at either the nucleic acid or protein level. Such methods are well known in the art and include but are not limited to nucleic acid hybridization techniques, nucleic acid reverse transcription methods, and nucleic acid amplification methods, western blots, northern blots, southern blots, ELISA, immunoprecipitation, immunofluorescence, flow cytometry, immunocytochemistry. In particular embodiments, disrupted gene transcription is detected on a protein level using, for example, antibodies that are directed against specific Cntnap2 or Auts2 proteins. These antibodies can be used in various methods such as Western blot, ELISA, immunoprecipitation, or immunocytochemistry techniques.

### I. Detection of Chromosomal Abnormalities and Sequence Variations

A number of assay formats known in the art are useful for detecting chromosomal abnormalities. These methods commonly involve nucleic acid binding, e.g., to filters, beads, or microliter plates and the like; and include dot-blot methods, Northern blots, Southern blots, PCR, and RFLP methods, and the like.

“Loci of interest” refers to a selected region of nucleic acid that is within a larger region of nucleic acid wherein the loci contains a chromosomal abnormality or a variant that contributes to the etiology of cognitive, behavioral, language, or social delays associated with ASD. In one embodiment, a loci of interest comprises any region of the CNTNAP2 gene. In another embodiment, a loci of interest comprises any region of the AUTS2 gene. A loci of interest can include, but is not limited to, 1-100, 1-50, 1-20, or 1-10 nucleotides, preferably 1-6, 1-5, 1-4, 1-3, 1-2, or 1 nucleotide(s).

The loci of interest can be analyzed by a variety of methods including but not limited to fluorescence detection, DNA sequencing gel, capillary electrophoresis on an automated DNA sequencing machine, microchannel electrophoresis, and other methods of sequencing, Sanger dideoxy sequencing, mass spectrometry, time of flight mass spectrometry, quadrupole mass spectrometry, magnetic sector mass spectrometry, electric sector mass spectrometry infrared spectrometry, ultraviolet spectrometry, palentiostatic amperometry or by DNA hybridization techniques including Southern Blot, Slot Blot, Dot Blot, and DNA microarray, wherein DNA fragments would be useful as both “probes” and “targets,” ELISA, fluorimetry, fluorescence polarization, Fluorescence Resonance Energy Transfer (FRET), SNP-IT, Gene Chips, HuSNP, BeadArray, TaqMan assay, Invader assay, MassExtend, or MassCleave™ (hMC) method.

### A. Karyotyping

Conventional procedures for genetic screening involve the analysis of karyotype. A karyotype is the particular chromosome complement of an individual or of a related group of individuals, as defined both by the number and morphology of the chromosomes usually in mitotic metaphase. It includes such things as total chromosome number, copy number of individual chromosome types (e.g., the number of copies of chromosome X), and chromosomal morphology, e.g., as measured by length, centromeric index, connectedness, or the like. Karyotypes are conventionally determined by chemically staining an organism's metaphase, prophase or otherwise condensed (for example, by premature chromosome condensation) chromosomes. Condensed chromosomes are used because, until recently, it has not been possible to visualize interphase chromosomes due to their dispersed condition and the lack of visible boundaries between them in the cell nucleus.

A number of cytological techniques based upon chemical stains have been developed which produce longitudinal patterns on condensed chromosomes, generally referred to as bands. The banding pattern of each chromosome within an organism usually permits unambiguous identification of each chromosome type (Latt, 1976, Annual Review of Biophysics and Bioengineering, 5: 1-37).

### B. Hybridization Assays

In one embodiment of the invention, chromosomal abnormalities are detected using a hybridization assay.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

(1) Fluorescence in situ hybridization (“FISH”) is a cytogenetic technique that can be used to detect and localize the presence or absence of specific DNA sequences on chromosomes (Verma et al., 1988, Human Chromosomes: A Manual Of Basic Techniques, Pergamon Press, New York). Fluorescent probes are used that only bind to those portions of a chromosome with which they share a high degree of sequence homology. FISH can also be used to detect and localize specific mRNAs within a tissue sample. of a cDNA clone to a metaphase chromosomal spread can be used to provide a precise chromosomal location in one step. This technique can be used with probes from the cDNA as short as 50 or 60 bp.

A FISH probe is constructed form fragments of isolated DNA and tagged directly with fluorophores, with targets for antibodies, or with biotin. Tagging can be done in various ways, for example nick translation and PCR using tagged nucleotides.

An interphase or metaphase chromosome preparation is produced from a sample obtained from a human subject. The chromosomes are firmly attached to a substrate, usually glass. Repetitive DNA sequences must be blocked by adding short fragments of DNA to the sample. The probe is then applied to the chromosome DNA and incubated for approximately 12 hours while hybridizing. Several wash steps remove all unhybridized or partially-hybridized probes. The results are then visualized and quantified using a microscope that is capable of exciting the dye and recording images.

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

(2) Allele specific hybridization can be used to detect pre-determined sequence variations, preferably a known mutation or set of known mutations in the test gene. In accordance with the invention, such pre-determined sequence variations are detected by allele specific hybridization, a sequence-dependent-based technique which permits discrimination between normal and mutant alleles. An allele specific assay is dependent on the differential ability of mismatched nucleotide sequences (e.g., normal:mutant) to hybridize with each other, as compared with matching (e.g., normal:normal or mutant:mutant) sequences.

A variety of methods well-known in the art can be used for detection of pre-determined sequence variations by allele specific hybridization. Preferably, the test gene is probed with allele specific oligonucleotides (ASOs); and each ASO contains the sequence of a known mutation. ASO analysis detects specific sequence variations in a target polynucleotide fragment by testing the ability of a specific oligonucleotide probe to hybridize to the target polynucleotide fragment. Preferably, the oligonucleotide contains the mutant sequence (or its complement). The presence of a sequence variation in the target sequence is indicated by hybridization between the oligonucleotide probe and the target fragment under conditions in which an oligonucleotide probe containing a normal sequence does not hybridize to the target fragment. A lack of hybridization between the sequence variant (e.g., mutant) oligonucleotide probe and the target polynucleotide fragment indicates the absence of the specific sequence variation (e.g., mutation) in the target fragment. In a preferred embodiment, the test samples are probed in a standard dot blot format. Each region within the test gene that contains the sequence corresponding to the ASO is individually applied to a solid surface, for example, as an individual dot on a membrane. Each individual region can be produced, for example, as a separate PCR amplification product using methods well-known in the art (see, for example, U.S. Pat. No. 4,683,202).

Membrane-based formats that can be used as alternatives to the dot blot format for performing ASO analysis include, but are not limited to, reverse dot blot, (multiplex amplification assay), and multiplex allele-specific diagnostic assay (MASDA).

In a reverse dot blot format, oligonucleotide or polynucleotide probes having known sequence are immobilized on the solid surface, and are subsequently hybridized with the labeled test polynucleotide sample. In this situation, the primers may be labeled or the NTPs may be labeled prior to amplification to prepare a labeled test polynucleotide sample. Alternatively, the test polynucleotide sample may be labeled subsequent to isolation and/or synthesis.

In a multiplex format, individual samples contain multiple target sequences within the test gene, instead of just a single target sequence. For example, multiple PCR products each containing at least one of the ASO target sequences are applied within the same sample dot. Multiple PCR products can be produced simultaneously in a single amplification reaction using the methods of Caskey et al., U.S. Pat. No. 5,582,989. The same blot, therefore, can be probed by each ASO whose corresponding sequence is represented in the sample dots.

A MASDA format expands the level of complexity of the multiplex format by using multiple ASOs to probe each blot (containing dots with multiple target sequences). This procedure is described in detail in U.S. Pat. No. 5,589,330 and in Michalowsky et al., 1996 (American Journal of Human Genetics, 59(4): A272, poster 1573) each of which is incorporated herein by reference in its entirety. First, hybridization between the multiple ASO probe and immobilized sample is detected. This method relies on the prediction that the presence of a mutation among the multiple target sequences in a given dot is sufficiently rare that any positive hybridization signal results from a single ASO within the probe mixture hybridizing with the corresponding mutant target. The hybridizing ASO is then identified by isolating it from the site of hybridization and determining its nucleotide sequence.

Suitable materials that can be used in the dot blot, reverse dot blot, multiplex, and MASDA formats are well-known in the art and include, but are not limited to nylon and nitrocellulose membranes.

When the target sequences are produced by PCR amplification, the starting material can be chromosomal DNA in which case the DNA is directly amplified. Alternatively, the starting material can be mRNA, in which case the mRNA is first reversed transcribed into cDNA and then amplified according to the well known technique of RT-PCR (see, for example, U.S. Pat. No. 5,561,058).

(3) Large scale arrays allow for the rapid analysis of many sequence variants. A review of the differences in the application and development of chip arrays is covered by Southern, 1996, Trends In Genetics 12: 110-115 and Cheng et al., 1996, Molecular Diagnosis, 1:183-200. Several approaches exist involving the manufacture of chip arrays. Differences include, but not restricted to: type of solid support to attach the immobilized oligonucleotides, labeling techniques for identification of variants and changes in the sequence-based techniques of the target polynucleotide to the probe.

A promising methodology for large scale analysis on ‘DNA chips’ is described in detail in Hacia et al., (Nature Genetics, 14:441-447) which is hereby incorporated by reference in its entirety. As described in Hacia et al., 1996, (Nature Genetics, 14:441-447) high density arrays of over 96,000 oligonucleotides, each 20 nucleotides in length, are immobilized to a single glass or silicon chip using light directed chemical synthesis. Contingent on the number and design of the oligonucleotide probe, potentially every base in a sequence can be interrogated for alterations. Oligonucleotides applied to the chip, therefore, can contain sequence variations that are not yet known to occur in the population, or they can be limited to mutations that are known to occur in the population.

Prior to hybridization with olignucleotide probes on the chip, the test sample is isolated, amplified and labeled (e.g. fluorescent markers) by means well known to those skilled in the art. The test polynucleotide sample is then hybridized to the immobilized oligonucleotides. The intensity of sequence-based techniques of the target polynucleotide to the immobilized probe is quantitated and compared to a reference sequence. The resulting genetic information can be used in molecular diagnosis.

A common, but not limiting, utility of the ‘DNA chip’ in molecular diagnosis is screening for known mutations. However, this may impose a limitation on the technique by only looking at mutations that have been described in the field. The present invention allows allele specific hybridization analysis be performed with a far greater number of mutations than previously available. Thus, the efficiency and comprehensiveness of large scale ASO analysis will be broadened, reducing the need for cumbersome end-to-end sequence analysis, not only with known mutations but in a comprehensive manner all mutations which might occur as predicted by the principles accepted, and the cost and time associated with these cumbersome tests will be decreased.

Array based comparative hybridization is another methodology that allows high resolution screening by hybridizing differentially labeled test and reference DNAs to arrays consisting of thousands of clones and detects chromosomal variations with high resolution.

### C. Amplification Assays

In one embodiment, chromosomal abnormalities are detected using an amplification assay. Template DNA can be amplified using any suitable method known in the art including but not limited to PCR (polymerase chain reaction), 3SR (self-sustained sequence reaction), LCR (ligase chain reaction), RACE-PCR (rapid amplification of cDNA ends), PLCR (a combination of polymerase chain reaction and ligase chain reaction), Q-beta phage amplification (Shah et al., J. Medical Micro. 33: 143541 (1995)), SDA (strand displacement amplification), SOE-PCR (splice overlap extension PCR), and the like. In a preferred embodiment, the template DNA is amplified using PCR (PCR: A Practical Approach, M. J. McPherson, et al., IRL Press (1991); PCR Protocols: A Guide to Methods and Applications, Innis, et al., Academic Press (1990); and PCR Technology: Principals and Applications of DNA Amplification, H. A. Erlich, Stockton Press (1989)). PCR is also described in numerous U.S. patents, including U.S. Pat. Nos. 4,683,195; 4,683,202; 4,800,159; 4,965,188; 4,889,818; 5,075,216; 5,079,352; 5,104,792, 5,023,171; 5,091,310; and 5,066,584.

1. Primer Design

Published sequences, including consensus sequences, can be used to design or select primers for use in amplification of template DNA. The selection of sequences to be used for the construction of primers that flank a locus of interest can be made by examination of the sequence of the loci of interest, or immediately thereto. The recently published sequence of the human genome provides a source of useful consensus sequence information from which to design primers to flank a desired human gene locus of interest.

By “flanking” a locus of interest is meant that the sequences of the primers are such that at least a portion of the 3′ region of one primer is complementary to the antisense strand of the template DNA and upstream from the locus of interest site (forward primer), and at least a portion of the 3′ region of the other primer is complementary to the sense strand of the template DNA and downstream of the locus of interest (reverse primer). A “primer pair” is intended a pair of forward and reverse primers. Both primers of a primer pair anneal in a manner that allows extension of the primers, such that the extension results in amplifying the template DNA in the region of the locus of interest.

Primers can be prepared by a variety of methods including but not limited to cloning of appropriate sequences and direct chemical synthesis using methods well known in the art (Narang et al., Methods Enzynol. 68:90 (1979); Brown et al., Methods Enzymol. 68:109 (1979)). Primers can also be obtained from commercial sources such as Operon Technologies, Amersham Pharmacia Biotech, Sigma, and Life Technologies. The primers can have an identical melting temperature. The lengths of the primers can be extended or shortened at the 5′ end or the 3′ end to produce primers with desired melting temperatures. In a preferred embodiment, one of the primers of the prime pair is longer than the other primer. In a preferred embodiment, the 3′ annealing lengths of the primers, within a primer pair, differ. Also, the annealing position of each primer pair can be designed such that the sequence and length of the primer pairs yield the desired melting temperature. The simplest equation for determining the melting temperature of primers smaller than 25 base pairs is the Wallace Rule (Td=2(A+T)+4(G+C)). Computer programs can also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineerin The TM (melting or annealing temperature) of each primer is calculated using software programs such as Net Primer (free web based program at http://premierbiosoft.com/netprimer/netprlaunch/netprlaunch.html; interne address as of Apr. 17, 2002).

In another embodiment, the annealing temperature of the primers can be recalculated and increased after any cycle of amplification, including but not limited to cycle 1, 2, 3, 4, 5, cycles 6-10, cycles 10-15, cycles 15-20, cycles 20-25, cycles 25-30, cycles 30-35, or cycles 35-40. After the initial cycles of amplification, the 5′ half of the primers is incorporated into the products from each loci of interest, thus the TM can be recalculated based on both the sequences of the 5′ half and the 3′ half of each primer.

As used herein, the term “about” with regard to annealing temperatures is used to encompass temperatures within 10° C. of the stated temperatures.

In one embodiment, one primer pair is used for each locus of interest. However, multiple primer pairs can be used for each locus of interest.

2. Template

Any nucleic acid specimen, in purified or nonpurified form, can be utilized as the starting nucleic acid or acids, providing it contains, or is suspected of containing, the specific nucleic acid sequence containing the CNTNAP2 gene, AUTS2 gene, or portions thereof. The term “template” therefore refers to any nucleic acid molecule that can be used for amplification in the invention. RNA or DNA that is not naturally double stranded can be made into double stranded DNA so as to be used as template DNA. Any double stranded DNA or preparation containing multiple, different double stranded DNA molecules can be used as template DNA to amplify a locus or loci of interest contained in the template DNA.

The template DNA can be from any appropriate sample including but not limited to, nucleic acid-containing samples of tissue, bodily fluid, umbilical cord blood, chorionic villi, amniotic fluid, an embryo, a two-celled embryo, a four-celled embryo, an eight-celled embryo, a 16-celled embryo, a 32-celled embryo, a 64-celled embryo, a 128-celled embryo, a 256-celled embryo, a 512-celled embryo, a 1024-celled embryo, embryonic tissues, lymph fluid, cerebrospinal fluid, mucosa secretion, or other body exudate, using protocols well established within the art.

In one embodiment, the template DNA can be obtained from a sample of a pregnant female. In another embodiment, the template DNA can be obtained from an embryo. In a preferred embodiment, the template DNA can be obtained from a single-cell of an embryo.

In one embodiment, the template DNA is fetal DNA. Fetal DNA can be obtained from sources including but not limited to maternal blood, maternal serum, maternal plasma, fetal cells, umbilical cord blood, chorionic villi, amniotic fluid, urine, saliva, cells or tissues.

The nucleic acid that is to be analyzed can be any nucleic acid, e.g., genomic, including DNA that has been reverse transcribed from an RNA sample, such as cDNA. The sequence of RNA can be determined according to the invention if it is capable of being made into a double stranded DNA form to be used as template DNA.

3. Amplification

The amplification step may amplify, for example, DNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid which contains one strand of each may be utilized. A mixture of nucleic acids may also be employed, or the nucleic acids produced in a previous amplification reaction herein, using the same or different primers may be so utilized. The specific nucleic acid sequence to be amplified, i.e., the polymorphic locus, may be a fraction of a larger molecule or can be present initially as a discrete molecule, so that the specific sequence constitutes the entire nucleic acid. It is not necessary that the sequence to be amplified be present initially in a pure form; it may be a minor fraction of a complex mixture, such as contained in whole human DNA.

In one embodiment, the nucleic acid is amplified directly in the original sample containing the source of nucleic acid. It is not essential that the nucleic acid be extracted, purified or isolated; it only needs to be provided in a form that is capable of being amplified. Hybridization of the nucleic acid template with primer, prior to amplification, is not required. For example, amplification can be performed in a cell or sample lysate using standard protocols well known in the art. DNA that is on a solid support, in a fixed biological preparation, or otherwise in a composition that contains non-DNA substances and that can be amplified without first being extracted from the solid support or fixed preparation or non-DNA substances in the composition can be used directly, without further purification, as long as the DNA can anneal with appropriate primers, and be copied, especially amplified, and the copied or amplified products can be recovered and utilized as described herein.

In a preferred embodiment, the nucleic acid is extracted, purified or isolated from non-nucleic acid materials that are in the original sample using methods known in the art prior to amplification.

In another embodiment, the nucleic acid is extracted, purified or isolated from the original sample containing the source of nucleic acid and prior to amplification, the nucleic acid is fragmented using any number of methods well known in the art including but not limited to enzymatic digestion, manual shearing, or sonication. For example, the DNA can be digested with one or more restriction enzymes that have a recognition site, and especially an eight base or six base pair recognition site, which is not present in the loci of interest. Typically, DNA can be fragmented to any desired length, including 50, 100, 250, 500, 1,000, 5,000, 10,000, 50,000 and 100,000 base pairs long. In another embodiment, the DNA is fragmented to an average length of about 1000 to 2000 base pairs. However, it is not necessary that the DNA be fragmented.

Fragments of DNA that contain the loci of interest can be purified from the fragmented DNA before amplification. Such fragments can be purified by using primers that will be used in the amplification (see “Primer Design” section below) as hooks to retrieve the loci of interest, based on the ability of such primers to anneal to the loci of interest. In a preferred embodiment, tag-modified primers are used, such as e.g. biotinylated primers.

By purifying the DNA fragments containing the loci of interest, the specificity of the amplification reaction can be improved. This will minimize amplification of nonspecific regions of the template DNA. Purification of the DNA fragments can also allow multiplex PCR (Polymerase Chain Reaction) or amplification of multiple loci of interest with improved specificity.

The components of a typical PCR reaction include but are not limited to a template DNA, primers, a reaction buffer (dependent on choice of polymerase), dNTPs (dATP, dTTP, dGTP, and dCTP) and a DNA polymerase. Suitable PCR primers can be designed and prepared according to methods well known in the art. Briefly, the reaction is heated to 95° C. for 2 minutes to separate the strands of the template DNA, the reaction is cooled to an appropriate temperature (determined by calculating the annealing temperature of designed primers) to allow primers to anneal to the template DNA, and heated to 72° C. for two minutes to allow extension.

After annealing, the temperature in each cycle is increased to an “extension” temperature to allow the primers to “extend” and then following extension the temperature in each cycle is increased to the denaturization temperature. For PCR products less than 500 base pairs in size, one can eliminate the extension step in each cycle and just have denaturization and annealing steps. A typical PCR reaction consists of 25-45 cycles of denaturation, annealing and extension as described above. However, as previously noted, one cycle of amplification (one copy) can be sufficient for practicing the invention.

In another embodiment, multiple sets of primers wherein a primer set comprises a forward primer and a reverser primer, can be used to amplify the template DNA for 1-5, 5-10, 10-15, 15-20 or more than 20 cycles, and then the amplified product is further amplified in a reaction with a single primer set or a subset of the multiple primer sets. In a preferred embodiment, a low concentration of each primer set is used to minimize primer-dimer formation. A low concentration of starting DNA can be amplified using multiple primer sets. Any number of primer sets can be used in the first amplification reaction including but not limiting to 1-10, 10-20, 20-30, 30-40, 40-50, 50-60, 60-70, 70-80, 80-90, 90-100, 100-150, 150-200, 200-250, 250-300, 300-350, 350-400, 400-450, 450-500, 500-1000, and greater than 1000. In another embodiment, the amplified product is amplified in a second reaction with a single primer set. In another embodiment, the amplified product is further amplified with a subset of the multiple primer pairs including but not limited to 2-10, 10-20, 20-30, 30-40, 40-50, 50-60, 60-70, 70-80, 80-90, 90-100, 100-150, 150-200, 200-250, and more than 250.

The multiple primer sets will amplify the loci of interest, such that a minimal amount of template DNA is not limiting for the number of loci that can be detected. For example, if template DNA is isolated from a single cell or the template DNA is obtained from a pregnant female, which comprises both maternal template DNA and fetal template DNA, low concentrations of each primer set can be used in a first amplification reaction to amplify the loci of interest. The low concentration of primers reduces the formation of primer-dimer and increases the probability that the primers will anneal to the template DNA and allow the polymerase to extend. The optimal number of cycles performed with the multiple primer sets is determined by the concentration of the primers. Following the first amplification reaction, additional primers can be added to further amplify the loci of interest. Additional amounts of each primer set can be added and further amplified in a single reaction. Alternatively, the amplified product can be further amplified using a single primer set in each reaction or a subset of the multiple primers sets. For example, if 150 primer sets were used in the first amplification reaction, subsets of 10 primer sets can be used to further amplify the product from the first reaction.

Any DNA polymerase that catalyzes primer extension can be used including but not limited to E. coli DNA polymerase, Klenow fragment of E. coli DNA polymerase 1, T7 DNA polymerase, T4 DNA polymerase, Taq polymerase, Pfu DNA polymerase, Vent DNA polymerase, bacteriophage 29, REDTaq™ Genomic DNA polymerase, or sequenase. Preferably, a thermostable DNA polymerase is used. A “hot start” PCR can also be performed wherein the reaction is heated to 95.degree. C. for two minutes prior to addition of the polymerase or the polymerase can be kept inactive until the first heating step in cycle 1. “Hot start” PCR can be used to minimize nonspecific amplification. Any number of PCR cycles can be used to amplify the DNA, including but not limited to 2, 5, 10, 15, 20, 25, 30, 35, 40, or 45 cycles. In a most preferred embodiment, the number of PCR cycles performed is such that equimolar amounts of each loci of interest are produced.

Purification of the amplified DNA is not necessary for practicing the invention. However, in one embodiment, if purification is preferred, the 5′ end of the primer (first or second primer) can be modified with a tag that facilitates purification of the PCR products. In a preferred embodiment, the first primer is modified with a tag that facilitates purification of the PCR products. The modification is preferably the same for all primers, although different modifications can be used if it is desired to separate the PCR products into different groups.

The tag can be any chemical moiety including but not limited to a radioisotope, fluorescent reporter molecule, chemiluminescent reporter molecule, antibody, antibody fragment, hapten, biotin, derivative of biotin, photobiotin, iminobiotin, digoxigenin, avidin, enzyme, acridinium, sugar, enzyme, apoenzyme, homopolymeric oligonucleotide, hormone, ferromagnetic moiety, paramagnetic moiety, diamagnetic moiety, phosphorescent moiety, luminescent moiety, electrochemiluminescent moiety, chromatic moiety, moiety having a detectable electron spin resonance, electrical capacitance, dielectric constant or electrical conductivity, or combinations thereof.

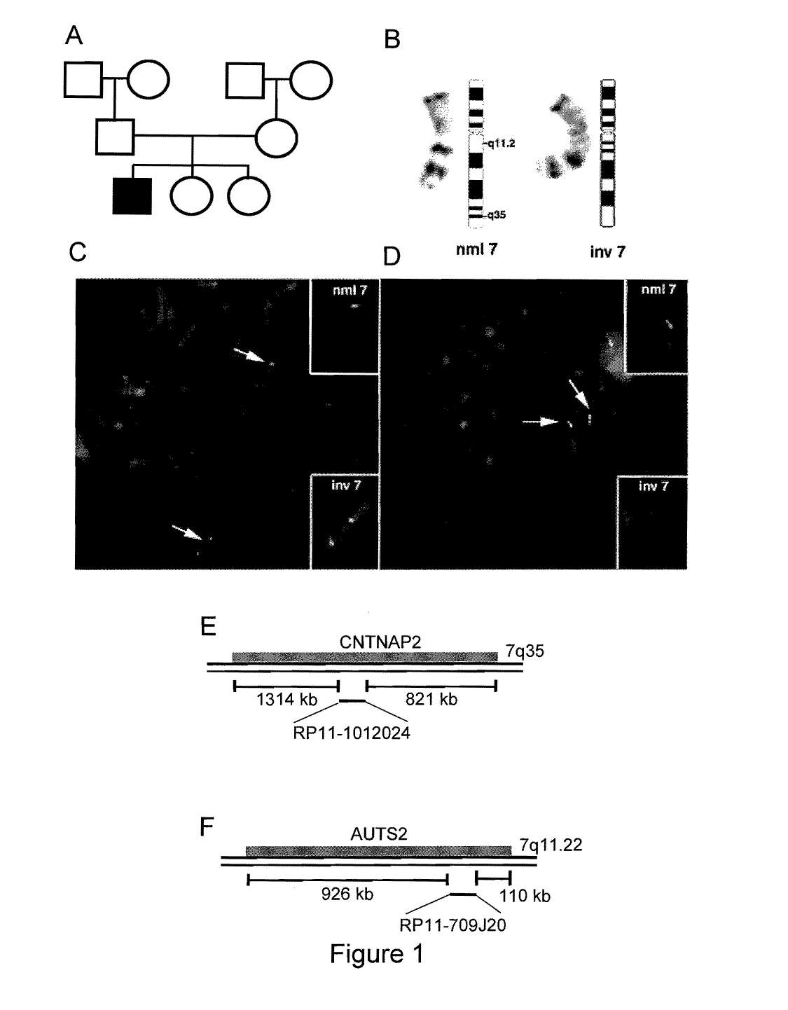

As one example, the 5′ ends of the primers can be biotinylated (Kandpal et al., Nucleic Acids Res. 18:1789-1795 (1990); Kaneoka et al., Biotechniques 10:30-34 (1991); Green et al., Nucleic Acids Res. 18:6163-6164 (1990)). The biotin provides an affinity tag that can be used to purify the copied DNA from the genomic DNA or any other DNA molecules that are not of interest. Biotinylated molecules can be purified using a streptavidin coated matrix as shown in FIG. 1F, including but not limited to Streptawell, transparent, High-Bind plates from Roche Molecular Biochemicals (catalog number 1 645 692, as listed in Roche Molecular Biochemicals, 2001 Biochemicals Catalog).

The PCR product of each locus of interest is placed into separate wells of a Streptavidin coated plate. Alternatively, the PCR products of the loci of interest can be pooled and placed into a streptavidin coated matrix, including but not limited to the Streptawell, transparent, High-Bind plates from Roche Molecular Biochemicals (catalog number 1 645 692, as listed in Roche Molecular Biochemicals, 2001 Biochemicals Catalog).

The amplified DNA can also be separated from the template DNA using non-affinity methods known in the art, for example, by polyacrylamide gel electrophoresis using standard protocols.

4. Sequence Analysis of Amplification Products

A variety of methods are employed to analyze the nucleotide sequence of the amplification products. Several techniques for detecting point mutations following amplification by PCR have been described in Chehab et al., 1992, Methods in Enzymology, 216:135-143; Maggio et al., 1993, Blood, 81(1):239-242; Cai and Kan, 1990, Journal of Clinical Investigation, 85(2):550-553; and Cai et al., 1989, Blood, 73:372-374.

One particularly useful technique is analysis of restriction enzyme sites following amplification. In this method, amplified nucleic acid segments are subjected to digestion by restriction enzymes. Identification of differences in restriction enzyme digestion between corresponding amplified segments in different individuals identifies a point mutation. Differences in the restriction enzyme digestion is commonly determined by measuring the size of restriction fragments by electrophoresis and observing differences in the electrophoretic patterns. Generally, the sizes of the restriction fragments is determined by standard gel electrophoresis techniques as described in Sambrook, et al, 2001, Molecular Cloning A Laboratory Manual, Cold Spring Harbor Press, and, e.g., in Polymeropoulos et al., 1992, Genomics, 12:492-496.

The size of the amplified segments obtained from affected and normal individuals and digested with appropriate restriction enzymes are analyzed on agarose or polyacrylamide gels. Because of the high discrimination of the polyacrylamide gel electrophoresis, differences of small magnitude are easily detected. Other mutations resulting in DPDD-related polymorphisms of DPD encoding genes also add unique restriction sites to the gene that are determined by sequencing DPDD-related nucleic acid sequences and comparing them to normal sequences.

Another useful method of identifying point mutations in PCR amplification products employs oligonucleotide probes specific for different sequences. The oligonucleotide probes are mixed with amplification products under hybridization conditions. Probes are either RNA or DNA oligonucleotides and optionally contain not only naturally occurring nucleotides but also analogs such as digoxygenin dCTP, biotin dCTP, 7-azaguanosine, azidothymidine, inosine, or uridine. The advantage of using nucleic acids comprising analogs include selective stability, resistance to nuclease activity, ease of signal attachment, increased protection from extraneous contamination and an increased number of probe-specific colored labels. For instance, in preferred embodiments, oligonucleotide arrays are used for the detection of specific point mutations as described below.

Probes are typically derived from cloned nucleic acids, or are synthesized chemically. When cloned, the isolated nucleic acid fragments are typically inserted into a replication vector, such as lambda phage, pBR322, M13, pJB8, c2RB, pcos1EMBL, or vectors containing the SP6 or 17 promoter and cloned as a library in a bacterial host. General probe cloning procedures are described in Sambrook, et al, 2001, Molecular Cloning A Laboratory Manual, Cold Spring Harbor Press.

The amplification products may also be detected by analyzing it by Southern blots without using radioactive probes. In such a process, for example, a small sample of DNA containing a very low level of the nucleic acid sequence of the polymorphic locus is amplified, and analyzed via a Southern blotting technique or similarly, using dot blot analysis. The use of non-radioactive probes or labels is facilitated by the high level of the amplified signal. Alternatively, probes used to detect the amplified products can be directly or indirectly detectably labeled, for example, with a radioisotope, a fluorescent compound, a bioluminescent compound, a chemiluminescent compound, a metal chelator or an enzyme. Those of ordinary skill in the art will know of other suitable labels for binding to the probe, or will be able to ascertain such, using routine experimentation. In the preferred embodiment, the amplification products are determinable by separating the mixture on an agarose gel containing ethidium bromide which causes DNA to be fluorescent.

Alternative methods of amplification have been described and can also be used in the practice of the instant invention. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA. Another nucleic acid amplification technique is nucleic acid sequence-based amplification (NASBA) which uses reverse transcription and T7 RNA polymerase and incorporates two primers to target its cycling scheme. NASBA can begin with either DNA or RNA and finish with either, and amplifies to 10⁸ copies within 60 to 90 minutes. Alternatively, nucleic acid can be amplified by ligation activated transcription (LAT). LAT works from a single-stranded template with a single primer that is partially single-stranded and partially double-stranded. Amplification is initiated by ligating a cDNA to the promoter olignucleotide and within a few hours, amplification is 10⁸ to 10⁹ fold. The QB replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest. Another nucleic acid amplification technique, ligase chain reaction (LCR), works by using two differently labeled halves of a sequence of interest which are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A 2-base gap separates the oligonucleotide probe pairs, and the RCR fills and joins the gap, mimicking normal DNA repair. Nucleic acid amplification by strand displacement activation (SDA) utilizes a short primer containing a recognition site for Hinc II with short overhang on the 5′ end which binds to target DNA. A DNA polymerase fills in the part of the primer opposite the overhang with sulfur-containing adenine analogs. Hinc II is added but only cuts the unmodified DNA strand. A DNA polymerase that lacks 5′ exonuclease activity enters at the cite of the nick and begins to polymerize, displacing the initial primer strand downstream and building a new one which serves as more primer. SDA produces greater than 10⁷-fold amplification in 2 hours at 37° C. Unlike PCR and LCR, SDA does not require instrumented Temperature cycling. Another amplification system useful in the method of the invention is the QB Replicase System.

### D. Sequencing Assays

In one embodiment, chromosomal abnormalities are detected using a sequencing assay. The term DNA sequencing encompasses biochemical methods for determining the order of the nucleotide bases, adenine, guanine, cytosine, and thymine, in a DNA molecule.

1. Chain-Termination Methods

The classical chain-termination or Sanger method requires a single-stranded DNA template, a DNA primer, a DNA polymerase, radioactively or fluorescently labeled nucleotides, and modified nucleotides that terminate DNA strand elongation. The DNA sample is divided into four separate sequencing reactions, containing all four of the standard deoxynucleotides (dATP, dGTP, dCTP and dTTP) and the DNA polymerase. To each reaction is added only one of the four dideoxynucleotides (ddATP, ddGTP, ddCTP, or ddTTP). These dideoxynucleotides are the chain-terminating nucleotides, lacking a 3′-OH group required for the formation of a phosphodiester bond between two nucleotides during DNA strand elongation. Incorporation of a dideoxynucleotide into the nascent (elongating) DNA strand therefore terminates DNA strand extension, resulting in various DNA fragments of varying length. The dideoxynucleotides are added at lower concentration than the standard deoxynucleotides to allow strand elongation sufficient for sequence analysis.

The newly synthesized and labeled DNA fragments are heat denatured, and separated by size (with a resolution of just one nucleotide) by gel electrophoresis on a denaturing polyacrylamide-urea gel. Each of the four DNA synthesis reactions is run in one of four individual lanes (lanes A, T, G, C); the DNA bands are then visualized by autoradiography or UV light, and the DNA sequence can be directly read off the X-ray film or gel image. In the image on the right, X-ray film was exposed to the gel, and the dark bands correspond to DNA fragments of different lengths. A dark band in a lane indicates a DNA fragment that is the result of chain termination after incorporation of a dideoxynucleotide (ddATP, ddGTP, ddCTP, or ddTTP). The terminal nucleotide base can be identified according to which dideoxynucleotide was added in the reaction giving that band. The relative positions of the different bands among the four lanes are then used to read (from bottom to top) the DNA sequence as indicated.

2. Dye-Terminator Sequencing

An alternative to primer labelling is labelling of the chain terminators, a method commonly called ‘dye-terminator sequencing’. The major advantage of this method is that the sequencing can be performed in a single reaction, rather than four reactions as in the labelled-primer method. In dye-terminator sequencing, each of the four dideoxynucleotide chain terminators is labelled with a different fluorescent dye, each fluorescing at a different wavelength. The dye-terminator sequencing method, along with automated high-throughput DNA sequence analyzers, is now being used for the vast majority of sequencing projects.

3. High-Throughput Sequencing

The high demand for low cost sequencing has given rise to a number of high-throughput sequencing technologies (Hall, 2007, The Journal of Experimental Biology 209: 1518-1525; Church, 2006, Scientific American 294: 47-54). Many of the new high-throughput methods use methods that parallelize the sequencing process, producing thousands or millions of sequences at once.

a. In Vitro Clonal Amplification

As molecular detection methods are often not sensitive enough for single molecule sequencing, most approaches use an in vitro cloning step to generate many copies of each individual molecule. Emulsion PCR is one method, isolating individual DNA molecules along with primer-coated beads in aqueous bubbles within an oil phase. A polymerase chain reaction (PCR) then coats each bead with clonal copies of the isolated library molecule and these beads are subsequently immobilized for later sequencing, also known as “emulsion PCR” (Margulies, et al., 2005, Nature 437: 376-380; Shendure, et al., 2005, Science 309:1728-1732).

Another method for in vitro clonal amplification is “bridge PCR”, where fragments are amplified upon primers attached to a solid surface, developed and used by Solexa. These methods both produce many physically isolated locations which each contain many copies of a single fragment. The single-molecule method developed by Stephen Quake's laboratory (later commercialized by Helicos) skips this amplification step, directly fixing DNA molecules to a surface.

b. Parallelized Sequencing

Once clonal DNA sequences are physically localized to separate positions on a surface, various sequencing approaches may be used to determine the DNA sequences of all locations, in parallel. “Sequencing by synthesis”, like the popular dye-termination electrophoretic sequencing, uses the process of DNA synthesis by DNA polymerase to identify the bases present in the complementary DNA molecule. Reversible terminator methods (used by Illumina and Helicos) use reversible versions of dye-terminators, adding one nucleotide at a time, detecting fluorescence corresponding to that position, then removing the blocking group to allow the polymerization of another nucleotide.

b.1 Sequencing by ligation is another enzymatic method of sequencing, using a DNA ligase enzyme rather than polymerase to identify the target sequence (Shendure et al., 2005, Science 309: 1728-1732; U.S. Pat. No. 5,750,341). This method uses a pool of all possible oligonucleotides of a fixed length, labeled according to the sequenced position. Oligonucleotides are annealed and ligated; the preferential ligation by DNA ligase for matching sequences results in a signal corresponding to the complementary sequence at that position.

b.2. Pyrosequencing is a method of DNA sequencing (determining the order of nucleotides in DNA) based on the “sequencing by synthesis” principle, which relies on detection of pyrophosphate release on nucleotide incorporation rather than chain termination with dideoxynucleotides (Margulies, et al., 2005, Nature 437:376-380; Ronaghi et al., 1996, Analytical Biochemistry 242:84-89).

“Sequencing by synthesis” involves taking a single strand of the DNA to be sequenced and then synthesizing its complementary strand enzymatically. The Pyrosequencing method is based on detecting the activity of DNA polymerase (a DNA synthesizing enzyme) with another chemiluminescent enzyme. Essentially, the method allows sequencing of a single strand of DNA by synthesizing the complementary strand along it, one base pair at a time, and detecting which base was actually added at each step. The template DNA is immobilized, and solutions of A, C, G, and T nucleotides are added and removed after the reaction, sequentially. Light is produced only when the nucleotide solution complements the first unpaired base of the template. The sequence of solutions which produce chemiluminescent signals allows the determination of the sequence of the template.

ssDNA template is hybridized to a sequencing primer and incubated with the enzymes DNA polymerase, ATP sulfurylase, luciferase and apyrase, and with the substrates adenosine 5′ phosphosulfate (APS) and luciferin. The addition of one of the four deoxynucleotide triphosphates (dNTPs) or, in the case of dATP, dATPaS, is added which is not a substrate for a luciferase) initiates the second step. DNA polymerase incorporates the correct, complementary dNTPs onto the template. This incorporation releases pyrophosphate (PPi) stoichiometrically. ATP sulfurylase quantitatively converts PPi to ATP in the presence of adenosine 5′ phosphosulfate. This ATP acts as fuel to the luciferase-mediated conversion of luciferin to oxyluciferin that generates visible light in amounts that are proportional to the amount of ATP. The light produced in the luciferase-catalyzed reaction is detected by a camera and analyzed in a program. Unincorporated nucleotides and ATP are degraded by the apyrase, and the reaction can restart with another nucleotide.

4. Other Sequencing Technologies

Other methods of DNA sequencing may have advantages in terms of efficiency or accuracy. Like traditional dye-terminator sequencing, they are limited to sequencing single isolated DNA fragments. “Sequencing by hybridization” is a non-enzymatic method that uses a DNA microarray. In this method, a single pool of unknown DNA is fluorescently labeled and hybridized to an array of known sequences. If the unknown DNA hybridizes strongly to a given spot on the array, causing it to “light up”, then that sequence is inferred to exist within the unknown DNA being sequenced. G. J. Hanna, V. A. Johnson, D. R. Kuritzkes, D. D. Richman, J. Martinez-Picado, L. Sutton, J. D. Hazelwood, R.T. D'Aquila, 2000, Journal of Clinical Microbiology 38 (7): 2715 Mass spectrometry can also be used to sequence DNA molecules; conventional chain-termination reactions produce DNA molecules of different lengths and the length of these fragments is then determined by the mass differences between them (rather than using gel separation; Edwards, et al. Mutation Research 573 (1-2): 3-12).

### II. Detection of a Disrupted Gene Product

### A. Protein Assays

In another embodiment of the invention, disruption of a gene product is detected at the protein level using antibodies specific for biomarker proteins of the invention. The method comprises obtaining a body sample from a patient, contacting the body sample with at least one antibody directed to a biomarker. One of skill in the art will recognize that the immunocytochemistry method described herein below is performed manually or in an automated fashion.

When the antibody used in the methods of the invention is a polyclonal antibody (IgG), the antibody is generated by inoculating a suitable animal with a biomarker protein, peptide or a fragment thereof. Antibodies produced in the inoculated animal which specifically bind the biomarker protein are then isolated from fluid obtained from the animal. Biomarker antibodies may be generated in this manner in several non-human mammals such as, but not limited to goat, sheep, horse, rabbit, and donkey. Methods for generating polyclonal antibodies are well known in the art and are described, for example in Harlow, et al. (1988, In: Antibodies, A Laboratory Manual, Cold Spring Harbor, N.Y.). These methods are not repeated herein as they are commonly used in the art of antibody technology.

When the antibody used in the methods of the invention is a monoclonal antibody, the antibody is generated using any well known monoclonal antibody preparation procedures such as those described, for example, in Harlow et al. (supra) and in Tuszynski et al. (1988, Blood, 72:109-115). Given that these methods are well known in the art, they are not replicated herein. Generally, monoclonal antibodies directed against a desired antigen are generated from mice immunized with the antigen using standard procedures as referenced herein. Monoclonal antibodies directed against full length or peptide fragments of biomarker may be prepared using the techniques described in Harlow, et al. (1988, In: Antibodies, A Laboratory Manual, Cold Spring Harbor, N.Y.).

Samples may need to be modified in order to render the biomarker antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO S1699)/dH2O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a biomarker of interest is then incubated with the sample.

Techniques for detecting antibody binding are well known in the art. Antibody binding to a biomarker of interest may be detected through the use of chemical reagents that generate a detectable signal that corresponds to the level of antibody binding and, accordingly, to the level of biomarker protein expression. In one of the preferred immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.), may be used to practice the present invention.

In one particular immunocytochemistry method of the invention, antibody binding to a biomarker is detected through the use of an HRP-labeled polymer that is conjugated to a secondary antibody. Antibody binding can also be detected through the use of a mouse probe reagent, which binds to mouse monoclonal antibodies, and a polymer conjugated to HRP, which binds to the mouse probe reagent. Slides are stained for antibody binding using the chromogen 3,3-diaminobenzidine (DAB) and then counterstained with hematoxylin and, optionally, a bluing agent such as ammonium hydroxide or TBS/Tween-20. In some aspects of the invention, slides are reviewed microscopically by a cytotechnologist and/or a pathologist to assess cell staining (i.e., biomarker overexpression). Alternatively, samples may be reviewed via automated microscopy or by personnel with the assistance of computer software that facilitates the identification of positive staining cells.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In regard to detection of antibody staining in the immunocytochemistry methods of the invention, there also exist in the art video-microscopy and software methods for the quantitative determination of an amount of multiple molecular species (e.g., biomarker proteins) in a biological sample, wherein each molecular species present is indicated by a representative dye marker having a specific color. Such methods are also known in the art as colorimetric analysis methods. In these methods, video-microscopy is used to provide an image of the biological sample after it has been stained to visually indicate the presence of a particular biomarker of interest. Some of these methods, such as those disclosed in U.S. patent application Ser. No. 09/957,446 and U.S. patent application Ser. No. 10/057,729 to Marcelpoil, incorporated herein by reference, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

The antibodies used to practice the invention are selected to have high specificity for the biomarker proteins of interest. Methods for making antibodies and for selecting appropriate antibodies are known in the art. See, for example, Celis, J. E. ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies of the invention may be selected on the basis of desirable staining of cytological, rather than histological, samples. That is, in particular embodiments the antibodies are selected with the end sample type (i.e., cytology preparations) in mind and for binding specificity.

One of skill in the art will recognize that optimization of antibody titer and detection chemistry is needed to maximize the signal to noise ratio for a particular antibody. Antibody concentrations that maximize specific binding to the biomarkers of the invention and minimize non-specific binding (or “background”) will be determined in reference to the type of biological sample being tested. In particular embodiments, appropriate antibody titers for use cytology preparations are determined by initially testing various antibody dilutions on formalin-fixed paraffin-embedded normal tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody is then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the invention will vary depending on such factors as time for binding, level of specificity of the antibody for the biomarker protein, and method of body sample preparation. Moreover, when multiple antibodies are used, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

### Immunoassays

Immunoassays, in their simplest and most direct sense, are binding assays. Certain preferred immunoassays are the various types of enzyme linked immunosorbent assays (ELISA) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, antibodies binding to the biomarker proteins of the invention are immobilized onto a selected surface exhibiting protein affinity, such as a well in a polystyrene microliter plate. Then, a test composition suspected of containing the biomarker antigen, such as a clinical sample, is added to the wells. After binding and washing to remove non-specifically bound immunecomplexes, the bound antibody may be detected. Detection is generally achieved by the addition of a second antibody specific for the target protein, that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the biomarker antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the biomarker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, the wells of the plate are incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating of nonspecific adsorption sites on the immobilizing surface reduces the background caused by nonspecific binding of antisera to the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to allow immunecomplex (antigen/antibody) formation” means that the conditions preferably include diluting the antigens and antibodies with solutions such as, but not limited to, BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this label is an enzyme that generates a color or other detectable signal upon incubating with an appropriate chromogenic or other substrate. Thus, for example, the first or second immunecomplex can be detected with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

B. mRNA Assays

In another embodiment of the invention, disruption of a gene product is detected at the mRNA level. Nucleic acid-based techniques for assessing mRNA expression are well known in the art and include, for example, determining the level of biomarker mRNA in a body sample. Many expression detection methods use isolated RNA. Any RNA isolation technique that does not select against the isolation of mRNA can be utilized for the purification of RNA from body samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155).

Isolated mRNA as a biomarker can be detected in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker of the present invention. Hybridization of an mRNA with the probe indicates that the biomarker in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an, alternative embodiment, the probe(s) are immobilized on a solid surface and the mRNA is contacted with the probe(s), for example, in an Affymetrix gene chip array (Santa Clara, Calif.). A skilled artisan can readily adapt known mRNA detection methods for use in detecting the level of mRNA encoded by the biomarkers of the present invention.

An alternative method for detecting biomarker mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189 193), self sustained sequence replication (Guatelli, 1990, Proc. Natl. Acad. Sci. USA, 87:1874 1878), transcriptional amplification system (Kwoh, 1989, Proc. Natl. Acad. Sci. USA, 86:1173 1177), Q-Beta Replicase (Lizardi, 1988, Bio/Technology, 6:1197), rolling circle replication (Lizardi, U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System). Such methods typically use pairs of oligonucleotide primers that are specific for the biomarker of interest. Methods for designing oligonucleotide primers specific for a known sequence are well known in the art.

Biomarker expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

### Kits

Kits for practicing the methods of the invention are further provided. By “kit” is intended any manufacture (e.g., a package or a container) comprising at least one reagent, e.g., an antibody, a nucleic acid probe, etc. for specifically detecting the expression of a biomarker of the invention. The kit may be promoted, distributed, or sold as a unit for performing the methods of the present invention. Additionally, the kits may contain a package insert describing the kit and including instructional material for its use.

Positive and/or negative controls may be included in the kits to validate the activity and correct usage of reagents employed in accordance with the invention. Controls may include samples, such as tissue sections, cells fixed on glass slides, etc., known to be either positive or negative for the presence of the biomarker of interest. The design and use of controls is standard and well within the routine capabilities of those of ordinary skill in the art.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials, methods and results of the experiments presented in this Example are now described.

### Example 1

### Mapping De Novo Inversion (inv(7)(q11.22;n35)) in a Child with Developmental Delay

A. Clinical Description of the (46,XY,inv(7)(q11.22;q35)) Patient

The patient is a 4.5-year-old male who was born at 38 weeks of gestation to his 33-year-old G3P3 mother by Caesarian section because of breech position. Birth weight was 3.3 kg. His neonatal course and infancy were complicated by poor feeding and severe gastresophageal reflux (confirmed by KUB/UGI at 2.5 months) in the context of global hypotonia. This eventually led to PEG tube placement at 6 months of age. Weight at 7 weeks was 4.4 kg (10th-25^{th} percentile). Genetic evaluation and testing at 3 months of age, in addition to a karyotype, included a normal FISH study for the Prader-Willi locus (SNRPN probe, 15q11.2), performed because of significant hypotonia. Antiviral antibody titers for toxoplasma, herpes simplex, and cytomegalovirus were negative at 2.5 months. Rubella IgG was 1.1 (at lower limit of immune range). Serum glucose and electrolytes were normal, with bicarbonate of 21 mEq/L and anion gap of 11. Urinalysis was normal, with no ketones. Lactic acid, at 3 months of age, was 1.4 (range 0.5-2.2) and ammonia was 63 (range 28-80). Creatine kinase level was 106 (normal range 0-200 IU/L). Hepatic transaminase values were within normal limits. Plasma amino acid and acylcarnitine analyses, and urine acylglycine and organic acid profiles, were normal. Transferrin isoelectric focusing to rule out carbohydrate-deficient glycoprotein syndromes was normal, as was plasma 7 dehydrocholesterol determination, to rule out Smith-Lemli-Opitz Syndrome. Cerebrospinal fluid amino acids, lactate, and pyruvate were normal. Ophthalmological evaluation at 3.5 months was initiated for a history of visual inattention during early infancy. Electro-retinogram and Preferential Looking Test of Visual Acuity were normal for age. Echocardiogram was normal at 7 months of age. Brain MRI at 2.5 months showed delayed myelination (lack of myelin within the anterior limb of the internal capsule, but normal myelination within the perirolandic white matter and posterior limbs of the internal capsules). In addition, there was a prominent subarachnoid space bifrontally with prominent ventricular system consistent with hypotrophy of the frontal and temporal lobes. EEG was normal.

Clinical genetic evaluation at 3.5 years revealed a past medical history significant for reflux in the first year of life, three previous episodes of pneumonia, hypotonia, tight heel cords, strabismus repair, and left inguinal hernia repair. He had pressure-equalizing tubes inserted into both ears for recurrent otitis media with conductive hearing loss. Family history was significant for two normally developing older siblings, and no history of cognitive or motor delays in an extended 3-generation pedigree. On physical examination, height was 100.2 cm (75th-90th percentile), weight was 14.7 kg (25th-50th percentile), and occipitofrontal head circumference was 49.4 cm (25th-50th percentile). Facies were essentially nondysmorphic except for surgically corrected strabismus and downslanting palpebral fissures. Distinctive physical findings included mild bilateral 5th digit clinodactyl), 2-3 toe syndactyl)-(not Y-shaped), genu and pes valgus, persistent fetal pads ontoes, tight Achille's tendons, and prominent scrotal raphe. Measurements of ocular distances, hands, feet, inter-nipple distance, and stretched penile length were within normal limits. No genetic syndrome was recognizable by his clinical geneticist (T.M.M.).

Developmentally, the patient did not smile socially until after 3 months, crawled at 13.5 months, walked and said his first word at 24 months, and began constructing 2-word phrases at 3 years of age. The Bayley Scales of Infant Development showed that the child was in the “significantly delayed” range. On the Vineland-II, a parent report instrument, the patient had the following standard scores (the mean for each test is 100 with a standard deviation of 15): communication, 67; daily living skills, 77; socialization, 77; motor, 64; and adaptive behavior composite, 68. Tests of fine motor skills with the Peabody Developmental Motor Scales-2 (PDMS-2) placed him 2 SD below the mean.

The patient was evaluated with the ADI-R and ADOS at the Yale Child Study Center at 49 months of age. On ADI-R, the parents reported an age at first word of 30 month and at first phrase of 48 months, which differs slightly from the documented medical history. Additionally, the parents reported that the patient had a “history of attacks that might be epileptic.” These, as noted, were followed up by a pediatrician with an EEG, which was normal. The patient met ADI-R scoring criteria for social (10), behavior (4), and age of onset (4). The patient did not meet cutoffs on the communication domains: verbal (0) or nonverbal (3). Based on the ADI-R algorithm used by AGRE repository (from which the mutation screening sample was derived), the patient would be classified as “Broad Spectrum.” However, the patient did not meet the ADOS criteria for a diagnosis of ASD.

### B. Results of Mapping Chromosomal Rearrangements Using Fluorescent In Situ Hybridization (FISH)

In order to detect chromosomal abnormalities present in an individual identified as having social and cognitive delays, G banded samples of metaphase chromosomes obtained from the above individual were prepared and probed using fluorescent in situ hybridization (FISH).

Inversion breakpoints disrupted the genes AUTS2 at 7q11.22 and CNTNAP2 at 7q35 in this individual (FIG. 1). AUTS2 maps to a 1.2 MB genomic region of 7q11.22; BAC RP11-709J20 spans the inversion and is within intron 5, placing the break between

exons 5 and 6. CNTNAP2 maps to a 2.3 MB genomic region on 7q35; BAC RP11-1012D24 was found to span the inversion and includes coding exons 11 and 12, placing
the break between exons 10 and 13. The patient was further evaluated by performing array-based competitive genomic hybridization with a chromosome 7-specific microarray
containing approximately 385,000 probes with an average spacing of 400 base pairs (Nimblegen). No largescale deletions or duplication were observed within several megabases of the breakpoints.

Both AUTS2 and CNTNAP2, either alone or in combination, are strong candidates for contributing to the etiology of the cognitive and social delays seen in the index case. AUTS2 encodes a predicted protein of unknown function that was originally identified through mapping of a chromosomal abnormality in a pair of twins with ASD (Sultana et al., 2002, Genomics 80:129-134). Additionally, three cases of MR and balanced translocations of AUTS2 have been reported (Kalscheuer et al., 2007, Human Genetics 121:501-509). However, a copy number polymorphism in unaffected individuals has also been reported at the AUTS2 locus (Redon et al., 2006, Nature 444:444-454), suggesting that haploinsufficiency and structural rearrangements at this interval may be tolerated in some cases. The expression of AUTS2 mRNA was evaluated by RT-PCR in peripheral lymphoblasts from the patient as well as unaffected family members; the patient's expression levels were normal for exons 50 to the break, but reduced by approximately 50% for exons distal to it (data not shown).

CNTNAP2 is also a strong candidate for involvement in social and cognitive delay. It is a neuronal cell adhesion molecule known to interact with Contactin 2 (Cntn2), also known as TAG-1, at the juxtaparanodal region at the nodes of Ranvier, which are the regularly spaced gaps between the myelin-producing Schwann cells in the

peripheral nervous system (PNS) (Traka et al., 2003, J. Cell Biol. 162:1161-1172; Poliak et al., 2003, J. Cell Biol. 162:1149-1160). Whereas previous investigations have largely focused on the role of CNTNAP2 in PNS development, a recent report demonstrated that
a homozygous CNTNAP2 mutation in the Old Order Amish population results in intractable seizures, histologically confirmed cortical neuronal migration abnormalities, MR, and ASD (Strauss et al., 2006, New Eng. J. Med. 354:1370-1377). These data, along with our earlier identification of a cytogenetic disruption of CNTN4 in a child with MR and ASD (Fernandez et al., 2004, Am. J. Human genetics 74:1286-1293), suggests the possible involvement of a Contactin-related pathway in these disorders.

As was the case with AUTS2, evidence from available reports of cytogenetic abnormalities involving CNTNAP2 has been inconsistent. In one instance, Tourette syndrome and developmental delay were identified in a family carrying a complex rearrangement disrupting CNTNAP2 (Verkerk et al., 2003, Genomics 82:1-9). More recently, carriers of a balanced t (Sebat et al., 2007, Science 316:445-449; Sultana et al., 2002, Genomics 80:129-134) translocation involving the coding region of CNTNAP2 were described as normal (Belloso et al., 2007, Eur. J. Hum. Genetics. 15:711-713. Given the absence of expression of CNTNAP2 in peripheral lymphoblasts, it was not possible to directly evaluate expression changes in the index case. However, the characterization of the de novo inversion described herein in the only affected member of the pedigree, coupled with previous findings with regard to CNTN4 (Fernandez et al., 2004, Am. J. Hum. Genetics 74:1286-1293) and the strong evidence that rare homozygous mutations in CNTNAP2 cause ASD3 support the hypothesis that this molecule plays a key role in central nervous system (CNS) development, and autism in particular.

### Example 2

### Expression of CNTNAP2/Cntnap2

### A. In Situ Hybridization

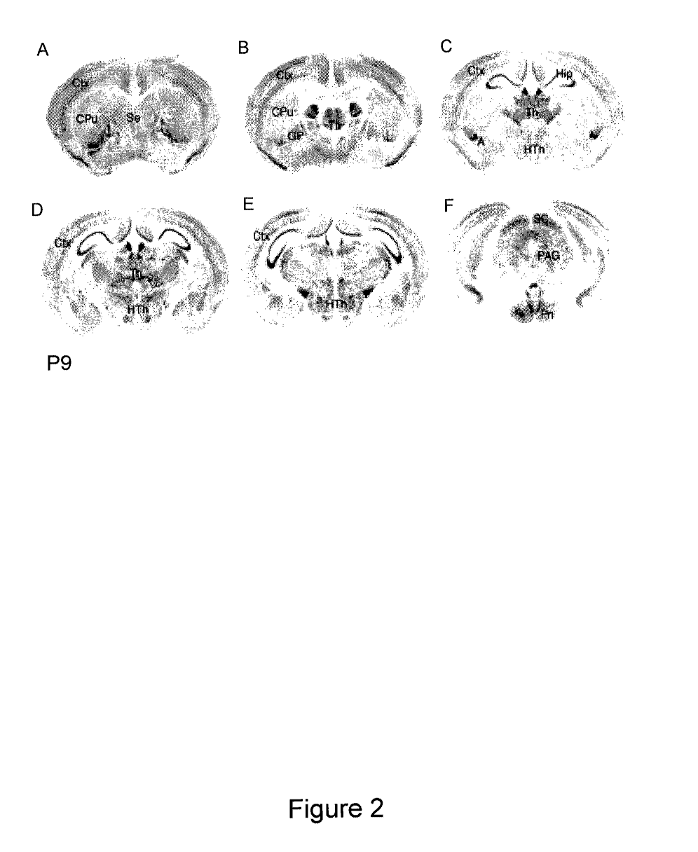

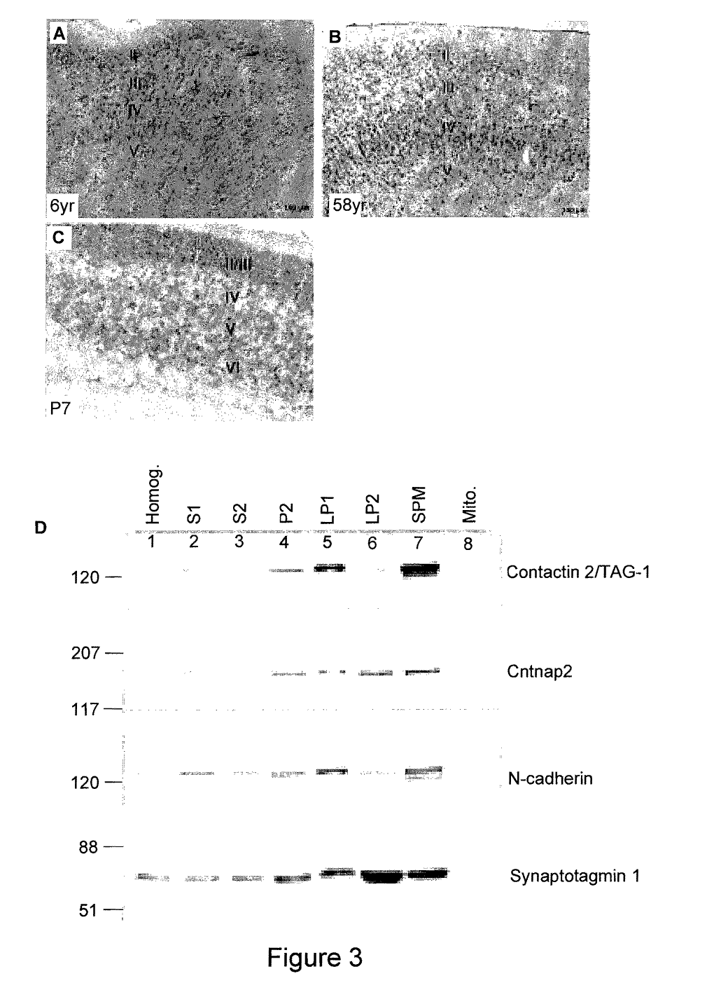

The distribution of Cntnap2 mRNA in the mouse and human CNS was examined by using in situ hybridization (Grove et al., 1998, Development 125:2315-2325) with digoxigenin-11-UTP RNA probes complementary to bases 3909 to 4890 of the mouse Cntnap2 cDNA (NM_{—}025771) or to bases 1343 to 2496 of the human CNTNAP2 cDNA (NM_{—}014141.3). Sections of P9 mouse brain were hybridized with a Cntnap2 antisense probe (FIG. 2). Sections of human temporal cortex at 6 and 58 years of age (FIG. 3A and FIG. 3B) and P7 mouse cortex (FIG. 3C) were also hybridized with corresponding antisense riboprobes.

### B. Rat Forebrain Subfractionation

Rat forebrain homogenate (homog.) was subfractionated into postnuclear supernatant (S1), synaptosomal supernatant (S2), crude synaptosomes (P2), synaptosomal membranes (LP1), crude synaptic vesicles (LP2), synaptic plasma membranes (SPM), and mitochondria (mito.) (FIG. 3D). The synaptic membrane protein N-cadherin and the synaptic vesicle protein synaptotagmin 1 served as markers for these respective fractions. Protein concentrations were determined with the Pierce BCA assay and equal amounts of each fraction were analyzed. Monoclonal antibodies to Cntn2/TAG-1 (3.1C12, developed by Thomas Jessell, Columbia University) were obtained from the Developmental Studies Hybridoma Bank maintained by the University of Iowa, to synaptotagmin 1 (41.1) from Synaptic Systems (Go\ddot{}ttingen, Germany), and to N-cadherin from 13D Biosciences (#610920). Polyclonal antibodies to Cntnap2 were obtained from Sigma (#C 8737).

C. Expression of CNTNAP2/Cntnap2 mRNA and Protein in Mouse and Human Central Nervous System

The distribution of Cntnap2 mRNA in the mouse and human CNS was examined by using in situ hybridization (Grove et al., 1998, Development 125:2315-2325) with digoxigenin-11-UTP RNA probes complementary to bases 3909 to 4890 of the mouse Cntnap2 cDNA (NM_{—}025771) or to bases 1343 to 2496 of the human CNTNAP2 cDNA (NM_{—}014141.3). Sections of P9 mouse brain were hybridized with a Cntnap2 antisense probe (FIG. 2).

Cntnap2 expression was detected in the cortex (FIG. 2A through FIG. 2D), septum (FIG. 2A), basal ganglia (FIG. 2A and FIG. 2B), many thalamic (FIG. 2B through FIG. 2D) and hypothalamic (FIG. 2C through FIG. 2E) nuclei, with particularly high levels observed in the anterior nucleus and the habenula, part of the amygdala (FIG. 2C), the superior colliculus and the periaqueductal gray (FIG. 2F), pons, cerebellum, and medulla, again with particularly high levels seen in the inferior olive.

Sections of human temporal cortex at 6 and 58 years of age (FIG. 3A and FIG. 3B) and P7 mouse cortex (FIG. 3C) were hybridized with corresponding antisense riboprobes. Expression is detected in cortical layers II-V in the human temporal lobe (FIG. 3A and FIG. 3B) and II-VI in the mouse neocortex (FIG. 3C). Widespread expression in embryonic and postnatal mouse brain was found including within the limbic system (FIGS. 2 and 3C), a neuroanatomical circuit implicated in social behavior. In human brain, previous findings of CNTNAP2 mRNA expression in all cortical layers of the temporal lobe was also confirmed (FIG. 3).

Cntnap2 protein expression and its putative binding partner, Cntn2/TAG-1, were also examined in subfractioned postnatal day 9 rat forebrain lysates (Jones and Matus, 1974, Biochem. Biophys. Acta 356:276-287; Biederer et al., 2002, Science 297:1525-1531). Both Cntnap2 and Cntn2/TAG-1 were present in the fraction containing synaptic plasma membranes, consistent with their forming a physical complex in this compartment (FIG. 3D). These data localized CNTNAP2 and elements of a Contactin-related pathway with neuronal structures of marked interest with regard to autism (Jamain et al., 2003, Nature Genetics 34:27-29; Laumonnier et al., 2004, Am. J. Hum. Genetics 74: 552-557; Zoghbi (2003) Science 302:826-830; Talebizadeh et al., 2004, J. Autism Dev. Disord. 34:735-736; Craig and Kang, 2007, Curr. Opin. Neurobio. 17:43-52; Durand et al., 2007, Nature genetics 39:25-27; Szatmari et al., 2007, Nature Genetics 39:25-27).

### Example 3

### Sequencing of CNTNAP2 Identifies Rare Unique Nonsynonymous Variants

### A. Subjects

The case group was comprised of affected children from 584 families that were obtained from the Autism Genetics Research Exchange (AGRE) and 51 affected children recruited at the Yale Child Study Center. Diagnoses included 96.7% autism, 2.0% broad spectrum, and 1.3% not quite autism (see AGRE diagnosis at http://agre.org/agrecatalog/algorithm.cfm). Males accounted for 81.1% of the sample. The ethnic/racial composition of the group was 587 white (92.4%), 24 white-Hispanic (3.8%), 7 unknown (1.1%), 6 Asian (0.9%), 6 more than one race (0.9%), 3 black or African-American (0.5%), 1 Native Hawaiian or Pacific Islander-Hispanic (0.2%), and 1 more than one race-Hispanic (0.2%). The resequenced control group consisted of 942 individuals: 757 white (80.4%), 94 white-Hispanic (10%), and 91 Asian (9.6%). These individuals were not evaluated for developmental delay or autism and were drawn from studies of renal disease, myocardial infarction, or normal human variation panels.

### B. DNA Re-Sequencing

DNA was amplified with a standard polymerase chain reaction (PCR) over 35 cycles with a 56.7° C. annealing temperature (Abelsom et al, 2005, Science 310:317-320) and analyzed with Sequencher (Genecodes) or PolyPhred software after dye terminating sequencing on one strand. Both cases and controls were evaluated in identical fashion in search of rare nonsynonymous, frame-shift, nonsense, and splice-site variants. Those changes that were found only in the case or the control group in the initial sequencing effort were further genotyped with Custom Taqman Genotyping assays (Applied Biosystems) in an additional control sample of 1073 unrelated white subjects. Variants with allele frequencies greater than 1/4000 in the combined control sample were excluded.

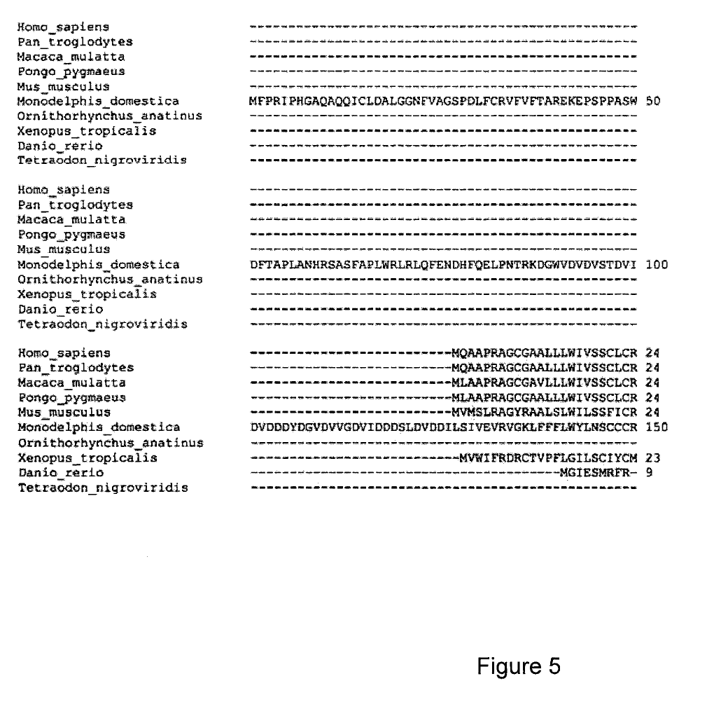

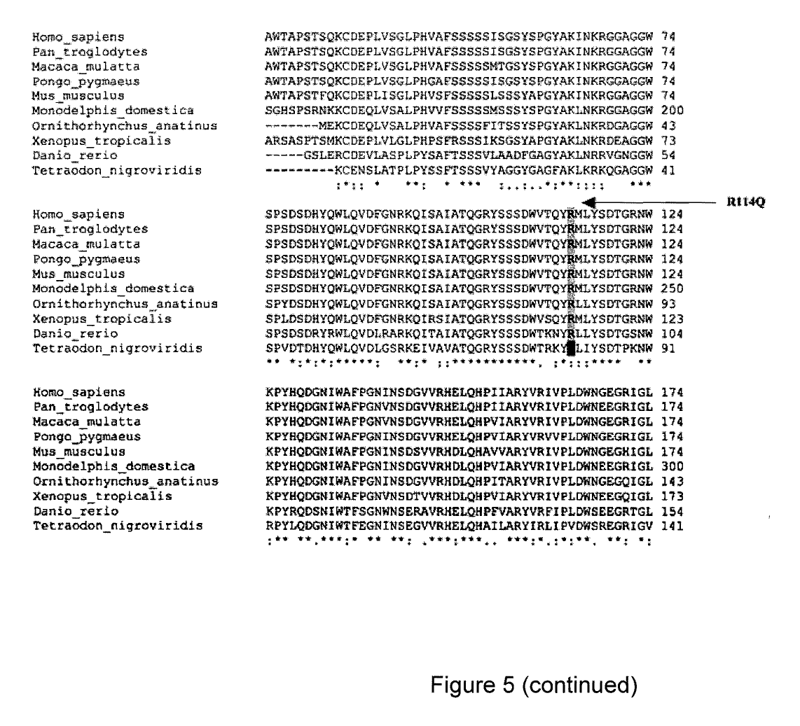

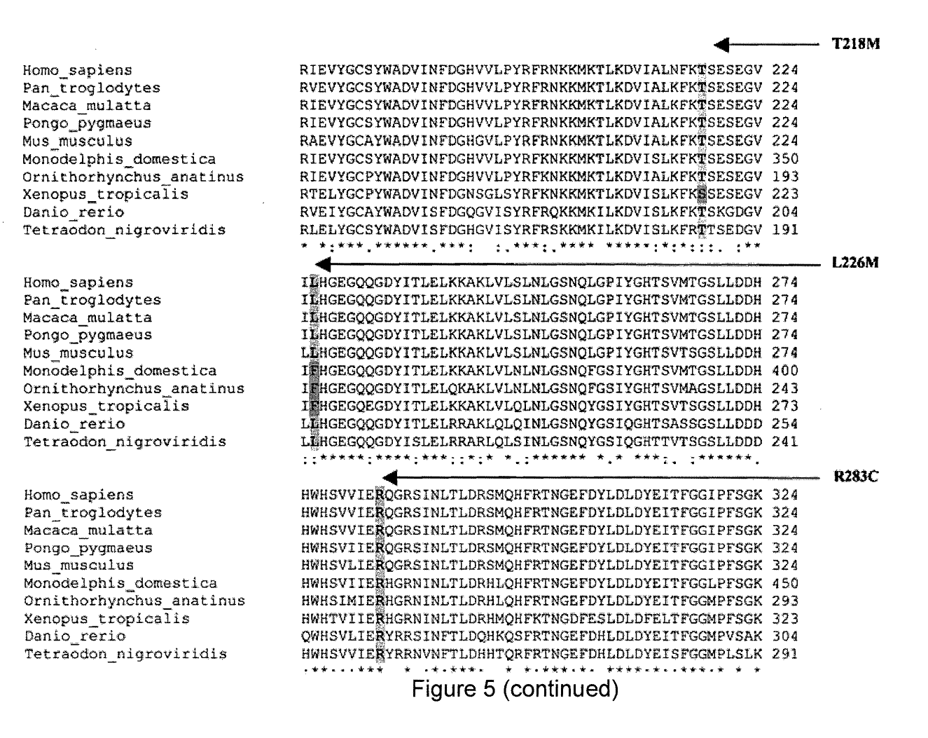

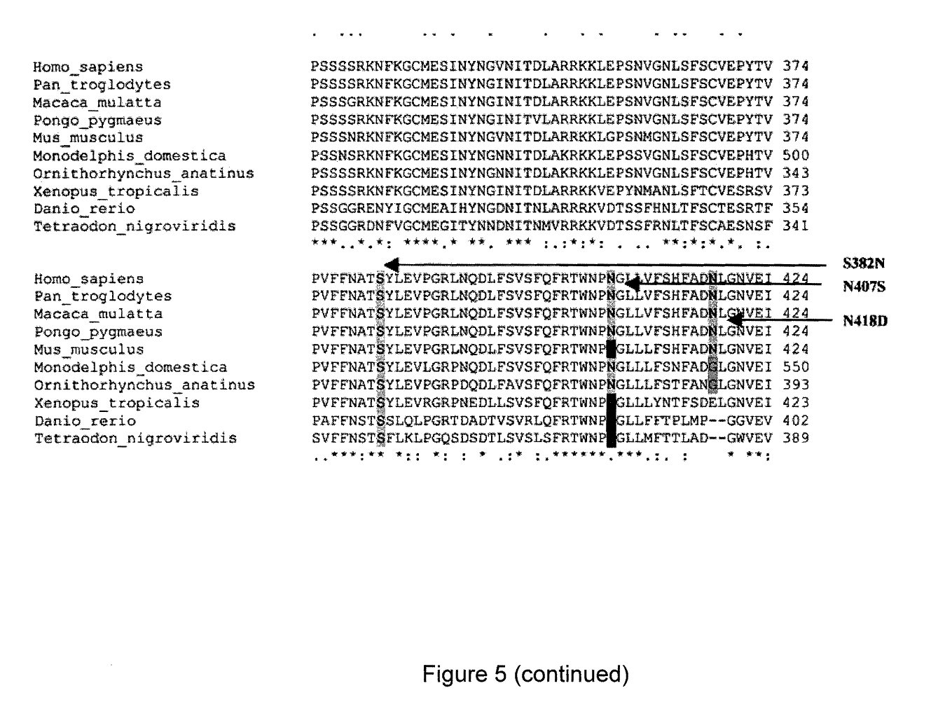

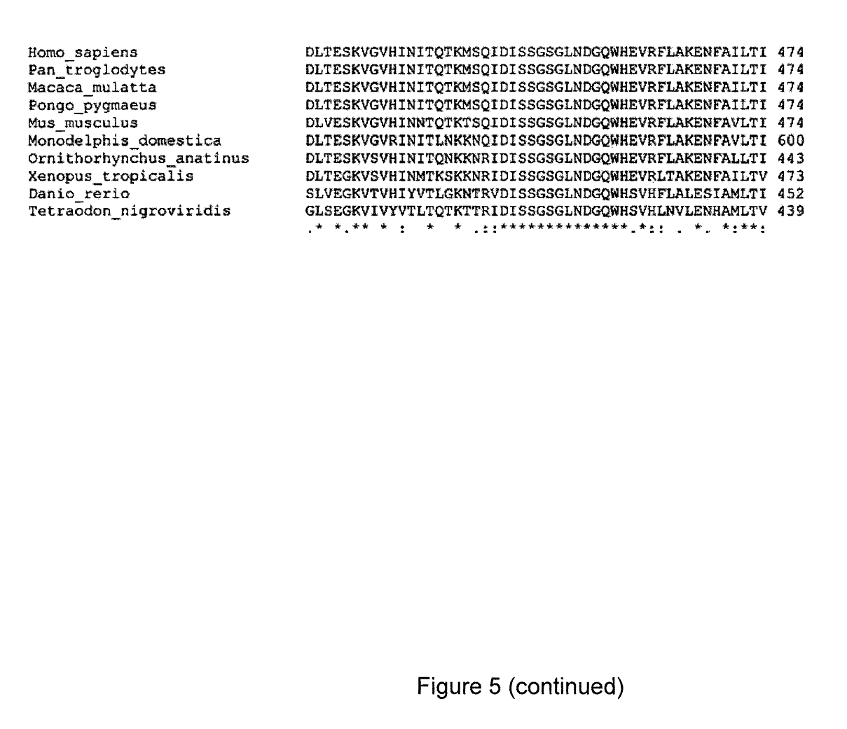

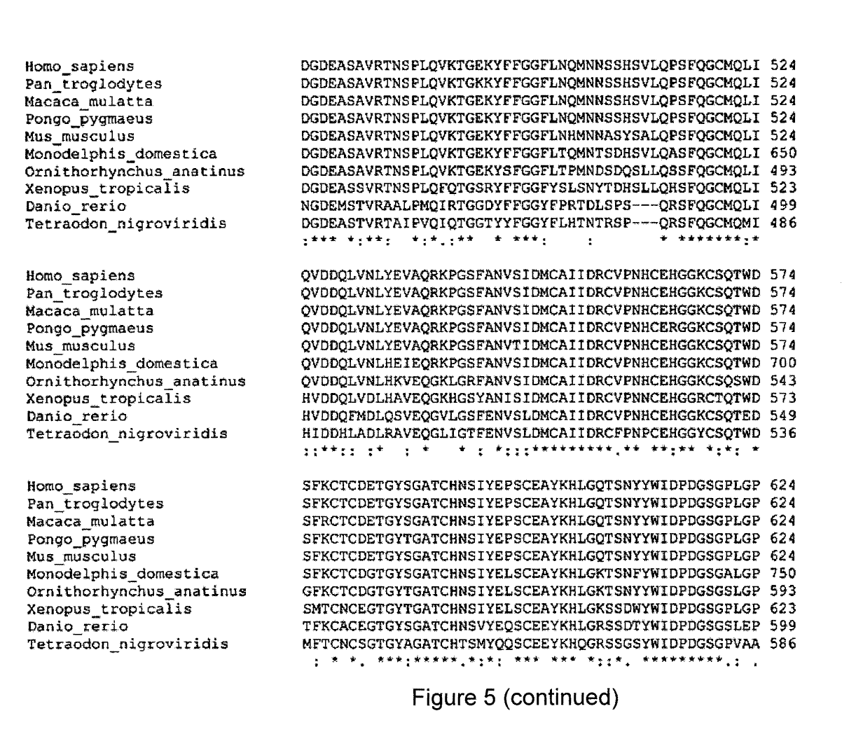

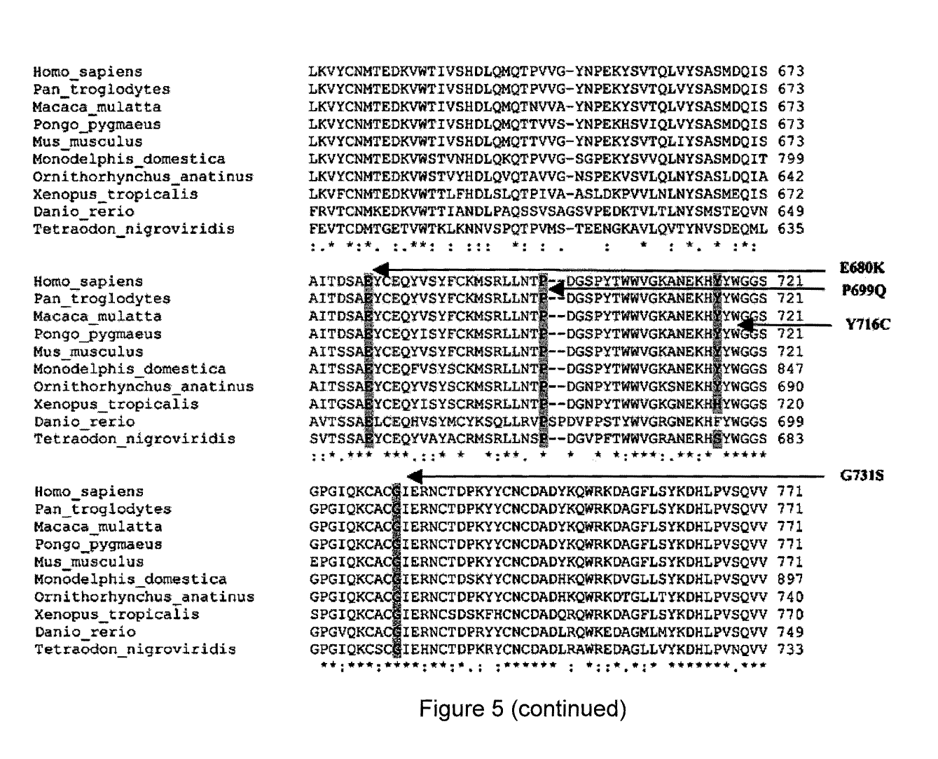

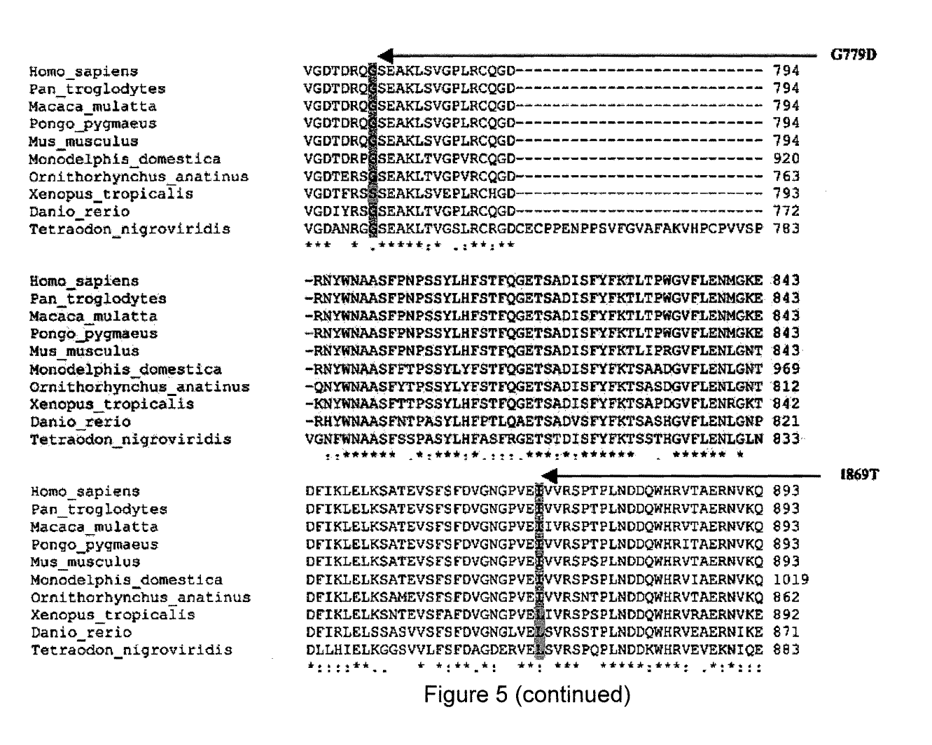

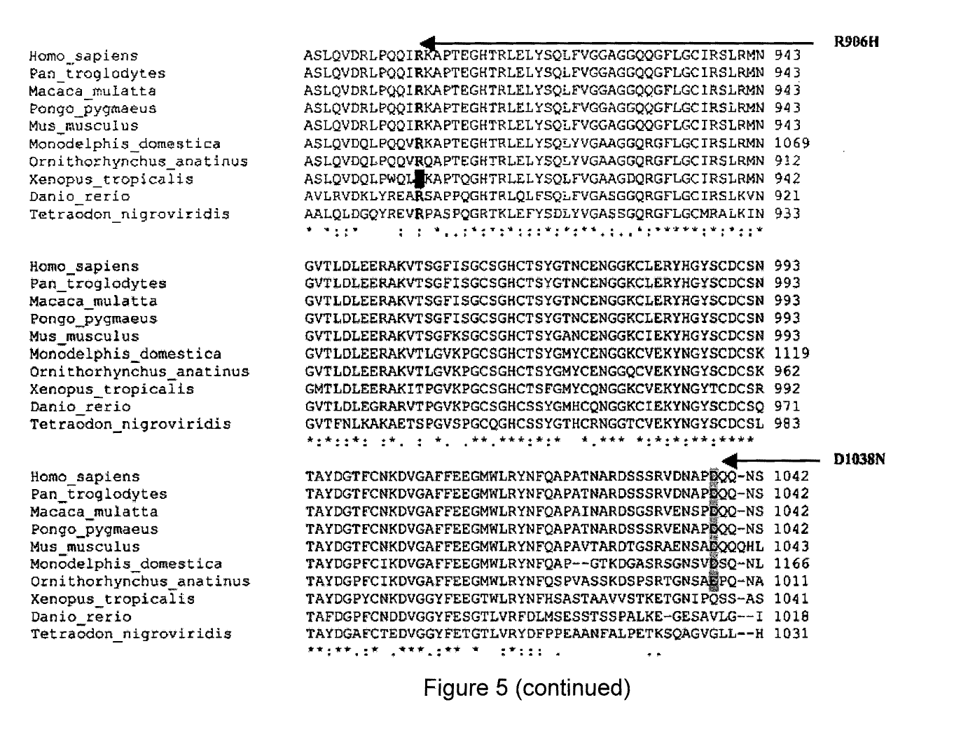

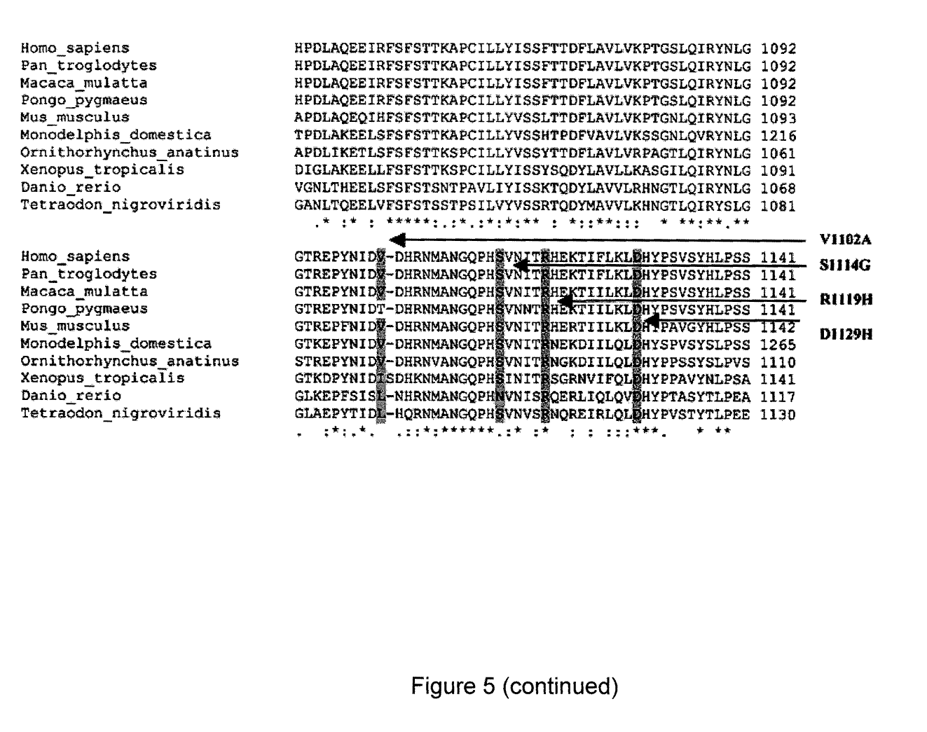

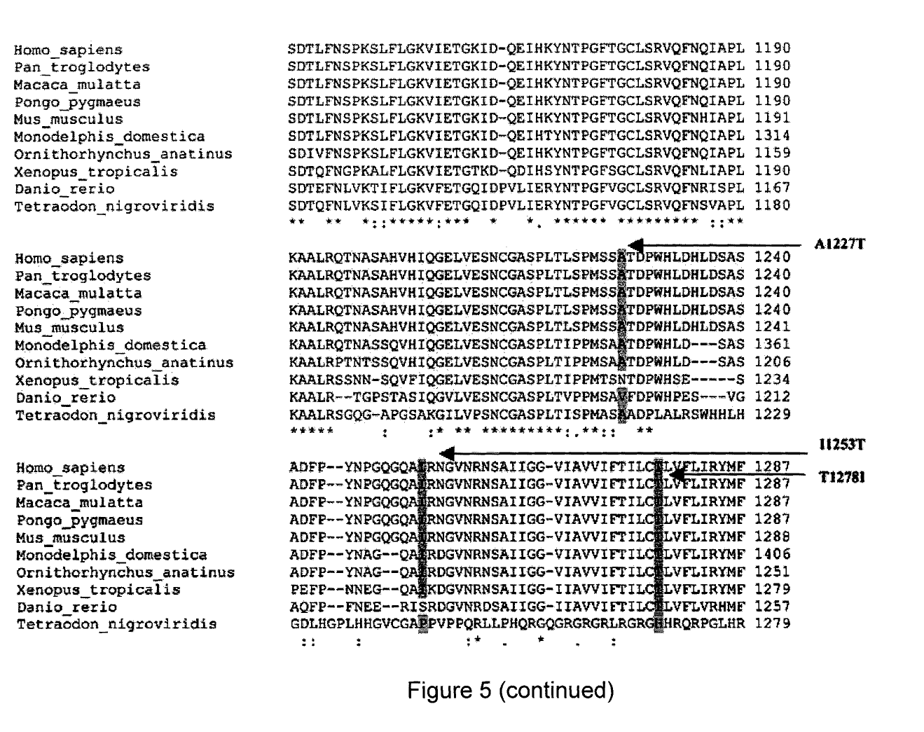

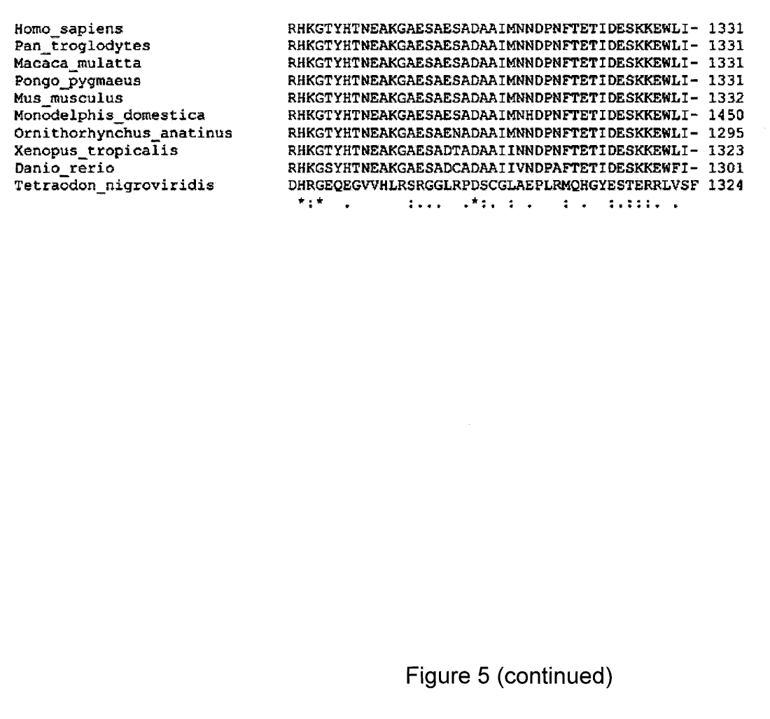

One variant, R283c, which was found once among the sequenced controls, failed further genotyping but was included in subsequent analyses. All rare nonsynonymous variants were examined for conservation across diverse species with a ClustalW alignment to the top full-length BLASTp hits of each species (Table 2 and FIG. 5). Additionally, substitutions were examined by the amino acid analysis programs Poly-Phen and SIFT (protein submission option), with Q9UHC6 as the reference CNTNAP2 protein, to identify those predicted to be possibly or probably deleterious to protein function (Table 2).

### C. Results of Resequencing of CNTNAP2

All 24 coding exons of CNTNAP2 were resequenced in 635 affected individuals and 942 uncharacterized controls (Table 1). This approach was selected because it is robust in the face of allelic heterogeneity and has proven valuable in identifying rare causal mutations in idiopathic autism (Jamain et al., 2003, Nature Genetics 34:27-29; Laumonnier et al., 2004, Am. J. Hum. Genetics 74:552-557). Moreover, in other complex genetic disorders, heterozygote nonsynonymous variants found in genes contributing to rare recessive diseases have been shown to confer risks in the broader population (Cohen et al., 2004, Science 305:869-872).

TABLE 1
Primer sequences for mutation screening of CNTNAP2

| Exon | Forward | SEQ ID | Reverse | SEQ ID | Product |
|---|---|---|---|---|---|
| no. | primer | NO. | primer | NO. | size (bp) |
| 1 | CACACAGTGCAAGAGGCAATAC | 9 | GATGCACTTCGGAGTTGATACC | 10 | 420 |
| 2 | TTAACCAACACATACCAATCGTT | 11 | GATTTCTGGTGTCTGCCAACAT | 12 | 298 |
| 3 | GAAATAGAGCACTGCCAAGACC | 13 | CATTGGATAGAAATTACAGCCTGA | 14 | 481 |
| 4 | ACCATTGGATGACATTTGTGTT | 15 | GGTAGTTTATTGTCAGAGAAAGCAA | 16 | 355 |
| 5 | CATTTATTCTTTGCAGACACCTG | 17 | TTTAAAGAATTGAGCAACATGAACA | 18 | 368 |
| 6 | TATCCCAGGTTAACTCGAATGG | 19 | TCAGGTTTTTAAAATTGTCAGTGTC | 20 | 466 |
| 7 | ATTTTGGAGGCAGAATGCTATAA | 21 | TTTTGCCCAAACACAAATATGAT | 22 | 400 |
| 8 | AGGCTGTGCTTCAAAACTTGTA | 23 | GTAACACCAGCAAAACCAAACA | 24 | 458 |
| 9 | AAATCGTGATTTGTTGATTTTGG | 25 | TTTTTGTTTTGCTCAGTGGAATTA | 26 | 382 |
| 10 | GTAGTTGGATGTGATGGCTGTG | 27 | TGGTAATTTCCACCTTACCTGTTT | 28 | 399 |
| 11 | ATATATTGCCCAGACAGCTTGG | 29 | TTGGTTTTTCAGATTCGAGTGA | 30 | 318 |
| 12 | GGTTTGCTAGCATTGCAATATG | 31 | GAAACAAACCATTGGTGGAACT | 32 | 292 |
| 13 | AACACTGTTCTACACCAGCTCAG | 33 | TCTTAGCTTCATTCCCCAGAAA | 34 | 496 |
| 14 | TCAGAGTATTCCTGGGGAAGTG | 35 | TTTGTCAGTTGGGTTAGTTCCA | 36 | 391 |
| 15 | TGCTATGAGACCACCTATGGAA | 37 | AGTCTGATTGCAGGCATCTTCT | 38 | 390 |
| 16 | GAGGATTTGGTCCAATGTTGTT | 39 | GGCTTGTGTGTCCACCTCTAGT | 40 | 465 |
| 17 | ATTTTGCCATCGACCTTTGTAG | 41 | TGTGCAGGCTCTTAAAAATCAAC | 42 | 468 |
| 18 | CTATGCAGTGTCATCTCCTACCAC | 43 | TTGGAAAATTCCTACCTAAGTTGA | 44 | 488 |
| 19 | ACTTACTCAGATGCCCTTCCTG | 45 | TGGCAAGTTGTTTTCCTGATATT | 46 | 539 |
| 20 | GACATCAAGGGAGGGAGTAAAG | 47 | CTATCCCCTCAAAACAAAACCA | 48 | 667 |
| 21 | GGTGTTTTAGAGTCAGTGCTGATG | 49 | AGAACAACCACGTAACTTTCCTGT | 50 | 381 |
| 22 | TGCAGCCCTAAATCTTATCGAC | 51 | CCTGAGAACTCCGTACTCACAA | 52 | 560 |
| 23 | CTGTTGTGATTCTTGTGGGAGA | 53 | CAGCAAAATGAATAATGTAAAAACC | 54 | 367 |
| 24 | CTGACGGAGCTGTAGTGAAGTG | 55 | CACGGGTCTTTAGAACACCTCTA | 56 | 611 |

^{a} As defined by NM_014141

TABLE 2
Unique Nonsynonymous Variants Identified
in ASD Cases and Controls

| | | | |
|---|---|---|---|
| Variant^{a} | Race/Ethnicity | Predicted Deleterious^{b} | Conserved^{c} |

ASD (n = 635)

| | | | |
|---|---|---|---|
| N4075^{d} | white | N | N |
| N418D | White-Hispanic | N | N |
| Y716C | white | N | N |
| G731S^{e,f} | >1 Asian | N | Y |
| I869T^{e} | white | Y, S | Y |
| I869T^{d,e} | white | Y, S | Y |
| I869T^{e} | white | Y, S | Y |
| R906H | white | N | N |
| R1119H^{e} | white | Y, P 7 S | Y |
| D1129H^{e} | White-Hispanic | Y, P & S | Y |
| A1227T | white | N | N |
| I1253T^{e} | White-Hispanic | Y, S | N |
| I1278I^{e} | white | Y, P & S | N |

Control (n = 942)

| | | | |
|---|---|---|---|
| R114Q | White-Hispanic | N | N |
| T218M^{e} | white | Y, P & S | Y |
| L226M^{e} | white | Y, S | Y |
| R283C^{e,g} | white | Y, P & S | Y |
| S382N^{e} | White-Hispanic | Y, S | Y |
| E680K^{e} | white | Y, P & S | Y |
| P699Q^{e} | White- Hispanic | N | Y |
| G779D | Asian | N | N |
| D1038N | white | N | N |
| V1102A | white | N | N |
| S114G | white | N | N |

^{a}Amino acid changes found only in cases (top of table) or only in controls (bottom of table)
^{b}P, PolyPhen; S, SIFT
^{c}Amino acids were considered conserved if all sequences were identical or only conserved substitutions were seen.
^{d}N407S/I869T were found in one proband on opposite chromosomes.
^{e}Variants predicted to be deleterious or conserved.
^{f}Parental DNA was sequenced and the suspect variant was determined to derive from the father who was Asian.
^{g}Variant failed genotyping

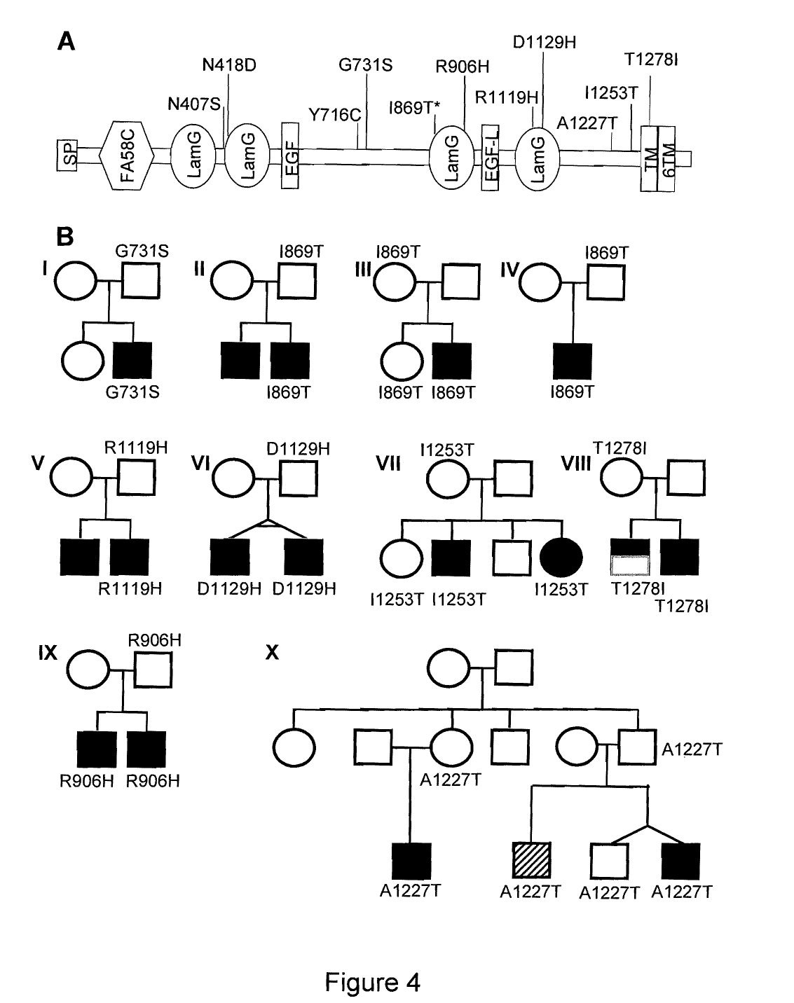

A total of 37 nonsynonymous variants were found among 645 cases, 23 of which had an allele frequency of less than 1/4000 (FIG. 4; Table 2 and Table 3). Of these 23 rare variants, 14 were predicted to be deleterious or were found at regions conserved across all species examined (FIG. 4A and FIG. 5).

In four cases, these potentially deleterious alleles were identified in pedigrees with more than one affected individual and three of these showed segregation with ASD in the affected first-degree relatives (FIG. 4B). Among the 942 controls, 35 nonsynonymous variants were identified; 11 of these were rare and 6 were predicted to be deleterious or were conserved across all species (FIG. 5; Table 2).

Table 3 presents ten additional rare variants present in the CNTNAP2 gene seen among 383 families with Autism.

TABLE 3

| | | Predicted | |
|---|---|---|---|
| Variant ^{a} | Affected individuals | Deleterious ^{b} | Conserved ^{c} |
| W134G ^{d} | Proband, father | yes | Yes |
| S287N | Proband, father, sibling | no | no |
| L292Q ^{d} | Proband, father | yes | yes |
| A545V | Proband, mother (sibling | no | partially |
| | unknown) | | |
| V708A ^{d} | Proband, mother, sibling 1 | yes | yes |
| | and sibling 2 | | |
| N735K ^{d} | Proband, mother | no | yes |
| T831S | Proband, father | no | no |
| Q921R ^{d} | Proband, father, sibling | yes | yes |
| R1027T ^{d} | Proband, father, sibling 1 | yes | no |
| | and sibling 2 | | |
| V1157A ^{d} | Proband, father | yes | yes |

^{a} Amino acid changes found only in cases (top of table) or only in controls (bottom of table)
^{b} determined by PolyPhen and SIFT
^{c} Amino acids were considered conserved if all sequences were identical or only conserved substitutions were seen.
^{d} Variants predicted to be deleterious or conserved.

Although the rates of all unique and predicted deleterious/conserved variants were, respectively, 135- and 2-fold higher in cases compared to controls, neither met a statistical threshold for an association of increased mutation burden with ASD (Fisher exact test p ¼ 0.21, OR 1.76 95% CI: 0.80-3.87; p ¼ 0.27, OR 1.98 95% CI: 0.72-5.49).

One highly conserved variant, I869T, which was predicted to be deleterious by SIFT, was identified in four affected individuals from three unrelated families with autism but was not present in 4010 control chromosomes, supporting an association for this substitution (Fisher exact test; p=0.014). In each family, the variant was inherited from an apparently unaffected parent. It was absence among several thousand control chromosomes, conserved across species, and segregated with affected status among first-degree relatives (FIG. 4B) all suggest that this variant warrants further attention.

When viewed in the context of two independent studies demonstrating linkage and/or association of common SNPs near CNTNAP2 with ASD (Alarcón al., 2008, Am. J. Hum. Genetics 82:150-159; Arking et al., 2008, Am. J. Hum. Genetics 82:160-164) these results both lend support to these findings and demonstrate the bounds of the potential contribution of rare variants in this transcript. Confirmation of the expression of CNTNAP2 in brain regions considered relevant in ASD as well as the demonstration of CNTNAP2 protein and its binding partner in the synaptic membrane support the biological plausibility of these findings, particularly given the identification of ASD-related mutations in other synaptic proteins including Neuroligin 3, Neuroligin 4 X-linked, SHANK3, and Neurexin 1 (Jamain et al., 2003, Nature Genetics 34:27-29; Laumonnier et al., 2004, Am. J. Hum. Genetics 74:552-557; Durand et al., 2007, Nature Genetics 39:25-27; Szatmari et al., 2007, Nature Genetics 39:319-328). The finding of a disrupted CNTNAP2 transcript resulting from a de novo chromosomal abnormality, the identification of multiple, rare, highly conserved variants in the case group that were not present in controls, and the association of I869T with ASD all suggest that some rare variants that disrupt protein function may contribute to disease risk.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of identifying a human subject at-risk of developing Autism Spectrum Disorder (ASD), said method comprising obtaining a body sample from said subject; detecting at least one chromosomal abnormality in a gene selected from the group consisting of the CNTNAP2 gene, the AUTS2 gene, and combinations thereof, wherein if at least one chromosomal abnormality is detected in said gene, then said subject is at-risk of developing ASD.

2. The method of claim 1, wherein said subject is selected from the group consisting of a fetus, a neonate, and a child.

3. The method of claim 2, wherein said child is less than or equal to 5 years old.

4. The method of claim 1, wherein said body sample is selected from the group consisting of a tissue, a cell, and a bodily fluid.

5. The method of claim 1, wherein said assay is selected from the group consisting of a PCR assay, a sequencing assay, an assay using a probe array, an assay using a gene chip, and an assay using a microarray.

6. A method of identifying a human subject at-risk of developing Autism Spectrum Disorder (ASD), said method comprising: obtaining a body sample from said subject; detecting at least one disrupted transcription of a gene selected from the group consisting of the CNTNAP2 gene, the AUTS2 gene, and combinations thereof, wherein if at least one disrupted transcript is detected in said gene, then said subject is at-risk of developing ASD.

7. The method of claim 6, wherein said method comprises an assay for mRNA selected from the group consisting of CNTNAP2 mRNA, AUTS2 mRNA, or a combination thereof.

8. The method of claim 7, wherein said assay comprises Northern blot analysis, in situ hybridization, or RT-PCR.

9. The method of claim 6, wherein said method comprises an assay for CNTNAP2 protein, AUTS2 protein, or a combination thereof.

10. The method of claim 9, where said assay comprises a Western blot analysis, radioimmunoassay (RIA), and immunoassay, chemiluminescent assay, or enzyme-linked immunosorbent assay (ELISA).

11. The method of claim 6, wherein said subject is selected from the group consisting of a fetus, a neonate, and a child.

12. The method of claim 11, wherein said child is less than or equal to 5 years old.

13. The method of claim 6, wherein said body sample is selected from the group consisting of a tissue, a cell, and a bodily fluid.

14. A method for determining in a human subject, the presence or absence of a sequence variation in a gene selected from the group consisting of CNTNAP2, AUTS2, or a combination thereof, said method comprising obtaining a body sample from said subject; detecting at least one sequence variation in a gene selected from the group consisting of the CNTNAP2 gene, the AUTS2 gene, and combinations thereof, wherein if at least one sequence variation is detected in either of said genes, then said subject is at-risk of developing ASD.

15. The method of claim 14, wherein said subject is selected from the group consisting of a fetus, a neonate, and a child.

16. The method of claim 15, wherein said child is less than or equal to 5 years old.

17. The method of claim 14, wherein said body sample is selected from the group consisting of a tissue, a cell, and a bodily fluid.

18. The method of claim 14, wherein said assay is selected from the group consisting of a PCR assay, a sequencing assay, an assay using a probe array, an assay using a gene chip, and an assay using a microarray.

19. The method of claim 14, wherein said sequence variation in said CNTNAP2 gene is selected from the group consisting of I869T, R1119H, D1129H, I1253T, I1278I, T218M, L226M, R283C, S382N, E680K, W134G, L292Q, V708A, Q921R, R1027T, and V1157A.

20. A method of identifying a human subject at-risk of germ-line transmission of Autism Spectrum Disorder (ASD) to progeny of said subject, said method comprising: obtaining a body sample from said subject; detecting at least one sequence variation of a gene selected from the group consisting of the CNTNAP2 gene, the AUTS2 gene, and combinations thereof, wherein if at least one sequence variation is detected in said gene, then said subject is at-risk of transmitting ASD to said progeny.

21. The method of claim 20, wherein said method comprises an assay for mRNA selected from the group consisting of CNTNAP2 mRNA, AUTS2 mRNA, or a combination thereof.

22. The method of claim 21, wherein said assay comprises Northern blot analysis, in situ hybridization, or RT-PCR.

23. The method of claim 20, wherein said method comprises an assay for CNTNAP2 protein, AUTS2 protein, or a combination thereof.

24. The method of claim 23, where said assay comprises a Western blot analysis, radioimmunoassay (RIA), and immunoassay, chemiluminescent assay, or enzyme-linked immunosorbent assay (ELISA).

25. The method of claim 20, wherein said body sample is selected from the group consisting of a tissue, a cell, and a bodily fluid.

26. The method of claim 20, wherein said sequence variation in said CNTNAP2 gene is selected from the group consisting of I869T, R1119H, D1129H, I1253T, I1278I, T218M, L226M, R283C, S382N, E680K, W134G, L292Q, V708A, Q921R, R1027T, and V1157A.

27. A method of prenatally identifying a human subject at-risk of germ-line transmission of Autism Spectrum Disorder (ASD) to progeny of said subject, said method comprising: obtaining a body sample from said subject; detecting at least one sequence variation of a gene selected from the group consisting of the CNTNAP2 gene, the AUTS2 gene, and combinations thereof, wherein if at least one sequence variation is detected in said gene, then said subject is at-risk of transmitting ASD to said progeny.

28. The method of claim 27, wherein said method comprises an assay for mRNA selected from the group consisting of CNTNAP2 mRNA, AUTS2 mRNA, or a combination thereof.

29. The method of claim 28, wherein said assay comprises Northern blot analysis, in situ hybridization, or RT-PCR.

30. The method of claim 27, wherein said method comprises an assay for CNTNAP2 protein, AUTS2 protein, or a combination thereof.

31. The method of claim 30, where said assay comprises a Western blot analysis, radioimmunoassay (RIA), and immunoassay, chemiluminescent assay, or enzyme-linked immunosorbent assay (ELISA).

32. The method of claim 27, wherein said body sample is selected from the group consisting of a tissue, a cell, and a bodily fluid.

33. The method of claim 27, wherein said sequence variation in said CNTNAP2 gene is selected from the group consisting of I869T, R1119H, D1129H, I1253T, I1278I, T218M, L226M, R283C, S382N, E680K, W134G, L292Q, V708A, Q921R, R1027T, and V1157A.
